SEQUENCE LISTING (25 sequences)

SEQ ID NO: 1 - DNA, 1350 bp, synthetic construct
  source 1..1350: mol_type = other DNA
atgggttctcatcatcatcatcatcatggtatggctagcatgactggtggacagcaaatgggtcgggatctgtacgacgatgacgataaggatctcgccaccatggtcgactcatcacgtcgtaagtggaataagacaggtcacgcagtcagagctataggtcggctgagctcactcgagaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagcttaagggcatcgacttcaaggaggacggcaacatcctggggcacaagctggagtacaacctgccggaccaactgactgaagagcagatcgcagaatttaaagagctgttctccctatttgacaaggacggggatgggacaataacaaccaaggagctggggacggtgatgcggtctctggggcagaaccccacagaagcagagctgcaggacatgatcaatgaagtagatgccgacggtgacggcacaatcgacttccctgagttcctgacaatgatggcaagaaaaatgaaatacacagacagtgaagaagaaattagagaagcgttccgtgtgtttgataaggatggcaatggctacatcagtgcagcagagcttcgccacgtgatgacaaaccttggagagaagttaacagatgaagaggttgatgaaatgatcagggaagcagacatcgatggggatggtcaggtaaactacgaagagtttgtacaaatgatgacagcgaag

SEQ ID NO: 2 - protein, 450 aa, synthetic construct
MGSHHHHHHGMASMTGGQQMGRDLYDDDDKDLATMVDSSRRKWNKTGHAVRAIGRLSSLENVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNLPDQLTEEQIAEFKELFSLFDKDGDGTITTKELGTVMRSLGQNPTEAELQDMINEVDADGDGTIDFPEFLTMMARKMKYTDSEEEIREAFRVFDKDGNGYISAAELRHVMTNLGEKLTDEEVDEMIREADIDGDGQVNYEEFVQMMTAK

SEQ ID NO: 3 - DNA, 708 bp, synthetic construct
  source 1..708: mol_type = other DNA
atggtgagcaagggcgaggaggataacatggccatcattaaggagttcatgcgcttcaaggtgcacatggaggggtccgtgaacggccacgtgttcgagatcgagggcgagggcgagggccgcccctacgagggcacccagaccgccaagctgaaggtgaccaagggtggccccctgcccttcacctgggacatcctgtcccctcaattcatgtacggctccaatgcctacgtgaagcaccccgccgacatccccgactactttaagctgtccttccccgagggcttcaagtgggagcgcgtgatgaaattcgaggacggcggcgtggtgaccgtgacccaggactcctccctgcaggacggtgagttcatctacaaggtgaagctgcgcggcaccaacttcccctccgacggccccgtaatgcagaagaagaccatgggctgggaggccctctccgagcggatgtaccccgaggacggcgccctgaagggcgaggtcaagccgagagtgaagctgaaggacggcggccactacgacgctgaggtcaagaccacctacaaggccaagaagcccgtgcagctgcccggcgcctacaacgtcaaccgcaagttggacatcacctcacacaacgaggactacaccatcgtggaacagtacgaacgtgccgagggccgccactccaccggcggcatggacgagctgtacaag

SEQ ID NO: 4 - protein, 236 aa, synthetic construct
MVSKGEEDNMAIIKEFMRFKVHMEGSVNGHVFEIEGEGEGRPYEGTQTAKLKVTKGGPLPFTWDILSPQFMYGSNAYVKHPADIPDYFKLSFPEGFKWERVMKFEDGGVVTVTQDSSLQDGEFIYKVKLRGTNFPSDGPVMQKKTMGWEALSERMYPEDGALKGEVKPRVKLKDGGHYDAEVKTTYKAKKPVQLPGAYNVNRKLDITSHNEDYTIVEQYERAEGRHSTGGMDELYK

SEQ ID NO: 5 - protein, 236 aa, synthetic construct
MVSKGEEDNMAIIKEFMRFKVHLEGSVNGHEFEIEGEGEGRPYEGTQTAKLKVTKGGPLPFAWDILSPQFMYGSNAYVKHPADIPDYFKLSFPEGFKWERVMNFEDGGVVTVTQDSSLQDGEFIYKVKLRGTNFPSDGPVMQKKTMGWETLSERMYPEDGALKGELKARTKLKDGGHYDTEVKTTYKAKKPVQLPGAYNVNRKLDITSHNEDYTIVEQYERAEGLHSTGGMDELYK

SEQ ID NO: 6 - protein, 236 aa, synthetic construct
MVSKGEEDNMAIIKEFMRFKVHLEGSVNGHEFEIEGEGEGRPYEGTQTAKLKVTKGGPLPFTWDILSPQFMYGSNAYVKHPADIPDYFKLSFPEGFKWERVMNFEDGGVVTVTQDSSLQDGEFIYKVKLRGTNFPSDGPVIQKKTMGWDALSERMYPEDGALKGELKARLKLKDGGHYEAEVKTTYKAKKPVQLPGAYNVNRKLDITSHNEDYTIVEQYERAEGRHSTGGMDELYK

SEQ ID NO: 7 - DNA, 57 bp, synthetic construct
  source 1..57: mol_type = other DNA
gccacgaacttctctctgttaaagcaagcaggagacgtggaagaaaaccccggtcct

SEQ ID NO: 8 - protein, 19 aa, synthetic construct
ATNFSLLKQAGDVEENPGP

SEQ ID NO: 9 - protein, 18 aa, synthetic construct
EGRGSLLTCGDVEENPGP

SEQ ID NO: 10 - protein, 20 aa, synthetic construct
QCTNYALLKLAGDVESNPGP

SEQ ID NO: 11 - protein, 22 aa, synthetic construct
VKQTLNFDLLKLAGDVESNPGP

SEQ ID NO: 12 - protein, 8 aa, synthetic construct
  VAR_SEQ 2: note = X is any amino acid
  VAR_SEQ 4: note = X is any amino acid
DXEXNPGP

SEQ ID NO: 13 - protein, 8 aa, synthetic construct
  VAR_SEQ 4: note = X is E or S
DVEXNPGP

SEQ ID NO: 14 - protein, 126 aa, synthetic construct
  VAR_SEQ 26: note = X is G or D
  VAR_SEQ 119: note = X is I or V
MPEPAKSAPAPKKGSKKAVTKAQKKXGKKRKRSRKESYSIYVYKVLKQVHPDTGISSKAMGIMNSFVNDIFERIAGEASRLAHYNKRSTITSREIQTAVRLLLPGELAKHAVSEGTKAXTKYTSAK

SEQ ID NO: 15 - DNA, 378 bp, synthetic construct
  source 1..378: mol_type = other DNA
atgccagagccagcgaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccatcaccaagtacaccagcgctaag

SEQ ID NO: 16 - protein, 126 aa, synthetic construct
MPEPAKSAPAPKKGSKKAVTKAQKKGGKKRKRSRKESYSIYVYKVLKQVHPDTGISSKAMGIMNSFVNDIFERIAGEASRLAHYNKRSTITSREIQTAVRLLLPGELAKHAVSEGTKAITKYTSAK

SEQ ID NO: 17 - protein, 126 aa, Homo sapiens [sequence omitted]

SEQ ID NO: 18 - protein, 6 aa, synthetic construct
DPPVAT

SEQ ID NO: 19 - protein, 4 aa, synthetic construct
AAAA

SEQ ID NO: 20 - protein, 8 aa, synthetic construct
DAAAAIAT

SEQ ID NO: 21 - DNA, 2541 bp, synthetic construct
  source 1..2541: mol_type = other DNA
atgggttctcatcatcatcatcatcatggtatggctagcatgactggtggacagcaaatgggtcgggatctgtacgacgatgacgataaggatctcgccaccatggtcgactcatcacgtcgtaagtggaataagacaggtcacgcagtcagagctataggtcggctgagctcactcgagaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagcttaagggcatcgacttcaaggaggacggcaacatcctggggcacaagctggagtacaacctgccggaccaactgactgaagagcagatcgcagaatttaaagagctgttctccctatttgacaaggacggggatgggacaataacaaccaaggagctggggacggtgatgcggtctctggggcagaaccccacagaagcagagctgcaggacatgatcaatgaagtagatgccgacggtgacggcacaatcgacttccctgagttcctgacaatgatggcaagaaaaatgaaatacacagacagtgaagaagaaattagagaagcgttccgtgtgtttgataaggatggcaatggctacatcagtgcagcagagcttcgccacgtgatgacaaaccttggagagaagttaacagatgaagaggttgatgaaatgatcagggaagcagacatcgatggggatggtcaggtaaactacgaagagtttgtacaaatgatgacagcgaagggagctactaacttcagcctgctgaagcaggctggagacgtggaggagaaccctggacctgatgctgctgctgctatcgccaccatgccagagccagcgaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccatcaccaagtacaccagcgctaaggatccaccggtcgccaccatggtgagcaagggcgaggaggataacatggccatcattaaggagttcatgcgcttcaaggtgcacatggaggggtccgtgaacggccacgtgttcgagatcgagggcgagggcgagggccgcccctacgagggcacccagaccgccaagctgaaggtgaccaagggtggccccctgcccttcacctgggacatcctgtcccctcaattcatgtacggctccaatgcctacgtgaagcaccccgccgacatccccgactactttaagctgtccttccccgagggcttcaagtgggagcgcgtgatgaaattcgaggacggcggcgtggtgaccgtgacccaggactcctccctgcaggacggtgagttcatctacaaggtgaagctgcgcggcaccaacttcccctccgacggccccgtaatgcagaagaagaccatgggctgggaggccctctccgagcggatgtaccccgaggacggcgccctgaagggcgaggtcaagccgagagtgaagctgaaggacggcggccactacgacgctgaggtcaagaccacctacaaggccaagaagcccgtgcagctgcccggcgcctacaacgtcaaccgcaagttggacatcacctcacacaacgaggactacaccatcgtggaacagtacgaacgtgccgagggccgccactccaccggcggcatggacgagctgtacaagtaa

SEQ ID NO: 22 - protein, 846 aa, synthetic construct
MGSHHHHHHGMASMTGGQQMGRDLYDDDDKDLATMVDSSRRKWNKTGHAVRAIGRLSSLENVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNLPDQLTEEQIAEFKELFSLFDKDGDGTITTKELGTVMRSLGQNPTEAELQDMINEVDADGDGTIDFPEFLTMMARKMKYTDSEEEIREAFRVFDKDGNGYISAAELRHVMTNLGEKLTDEEVDEMIREADIDGDGQVNYEEFVQMMTAKGATNFSLLKQAGDVEENPGPDAAAAIATMPEPAKSAPAPKKGSKKAVTKAQKKGGKKRKRSRKESYSIYVYKVLKQVHPDTGISSKAMGIMNSFVNDIFERIAGEASRLAHYNKRSTITSREIQTAVRLLLPGELAKHAVSEGTKAITKYTSAKDPPVATMVSKGEEDNMAIIKEFMRFKVHMEGSVNGHVFEIEGEGEGRPYEGTQTAKLKVTKGGPLPFTWDILSPQFMYGSNAYVKHPADIPDYFKLSFPEGFKWERVMKFEDGGVVTVTQDSSLQDGEFIYKVKLRGTNFPSDGPVMQKKTMGWEALSERMYPEDGALKGEVKPRVKLKDGGHYDAEVKTTYKAKKPVQLPGAYNVNRKLDITSHNEDYTIVEQYERAEGRHSTGGMDELYK

SEQ ID NO: 23 - DNA, 365 bp, Homo sapiens
  source 1..365: mol_type = other DNA
cacttgtggactaagtttgttcgcatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagccacagtgccctgctcagaagccccaagctcgtcagtcaagccggttctccgtttgcactcaggagcacgggcaggcgagtggcccctagttctgggggcagc

SEQ ID NO: 24 - DNA, 1104 bp, synthetic construct
  source 1..1104: mol_type = other DNA
atgccagagccagcgaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccatcaccaagtacaccagcgctaaggatccaccggtcgccaccatggtgagcaagggcgaggaggataacatggccatcattaaggagttcatgcgcttcaaggtgcacatggaggggtccgtgaacggccacgtgttcgagatcgagggcgagggcgagggccgcccctacgagggcacccagaccgccaagctgaaggtgaccaagggtggccccctgcccttcacctgggacatcctgtcccctcaattcatgtacggctccaatgcctacgtgaagcaccccgccgacatccccgactactttaagctgtccttccccgagggcttcaagtgggagcgcgtgatgaaattcgaggacggcggcgtggtgaccgtgacccaggactcctccctgcaggacggtgagttcatctacaaggtgaagctgcgcggcaccaacttcccctccgacggccccgtaatgcagaagaagaccatgggctgggaggccctctccgagcggatgtaccccgaggacggcgccctgaagggcgaggtcaagccgagagtgaagctgaaggacggcggccactacgacgctgaggtcaagaccacctacaaggccaagaagcccgtgcagctgcccggcgcctacaacgtcaaccgcaagttggacatcacctcacacaacgaggactacaccatcgtggaacagtacgaacgtgccgagggccgccactccaccggcggcatggacgagctgtacaag

SEQ ID NO: 25 - protein, 368 aa, synthetic construct
MPEPAKSAPAPKKGSKKAVTKAQKKGGKKRKRSRKESYSIYVYKVLKQVHPDTGISSKAMGIMNSFVNDIFERIAGEASRLAHYNKRSTITSREIQTAVRLLLPGELAKHAVSEGTKAITKYTSAKDPPVATMVSKGEEDNMAIIKEFMRFKVHMEGSVNGHVFEIEGEGEGRPYEGTQTAKLKVTKGGPLPFTWDILSPQFMYGSNAYVKHPADIPDYFKLSFPEGFKWERVMKFEDGGVVTVTQDSSLQDGEFIYKVKLRGTNFPSDGPVMQKKTMGWEALSERMYPEDGALKGEVKPRVKLKDGGHYDAEVKTTYKAKKPVQLPGAYNVNRKLDITSHNEDYTIVEQYERAEGRHSTGGMDELYK

(19) United States
(12) Patent Application Publication
(10) Pub. No.: US 2025/0303003 A1
(43) Pub. Date: Oct. 2, 2025

(54) METHODS OF ON DEMAND IN VIVO PHOTOTAGGING

(71) Applicants: YEDA RESEARCH AND DEVELOPMENT CO. LTD., Rehovot (IL); THE TRUSTEES OF COLUMBIA UNIVERSITY IN THE CITY OF NEW YORK, New York, NY (US)

(72) Inventors: Ivo SPIEGEL, Tel-Aviv (IL); Dahlia KUSHINSKY, Rehovot (IL); Boaz NUTKOVICH, Rehovot (IL); Attila LOSONCZY, New York, NY (US); Jingcheng SHI, New York, NY (US)

(21) Appl. No.: 19/090,700

(22) Filed: Mar. 26, 2025

Related U.S. Application Data
(60) Provisional application No. 63/569,772, filed on Mar. 26, 2024.

(51) Int. Cl. A61K 49/00; C07K 7/08; C07K 14/47; C12N 15/86; C12Q 1/24; C12Q 1/6869

(52) U.S. Cl. CPC A61K 49/0045; C07K 7/08; C07K 14/4716; C12N 15/86; C12Q 1/24; C12Q 1/6869; C07K 2319/60; C12N 2750/14143; C12N 2830/008

(57) ABSTRACT

Nucleic acid molecules comprising at least one transcription regulatory element operably linked to an open reading frame, wherein the open reading frame encodes a single RNA transcript encoding GCaMP7f, a ribosomal skipping peptide, and a fusion protein of a nuclear protein and photoactivatable red fluorescent protein are provided. Expression vectors and cells comprising the nucleic acid molecules are also provided, as are methods of using the nucleic acid molecules for simultaneous labeling and measuring calcium and analyzing a target cell.

### CROSS REFERENCE TO RELATED APPLICATIONS

This application claims the benefit of priority of U.S. Provisional Patent Application No. 63/569,772 filed on Mar. 26, 2024, the contents of which are all incorporated herein by reference in their entirety.

### REFERENCE TO AN ELECTRONIC SEQUENCE LISTING

The contents of the electronic sequence listing (YEDA-CU-P-053-US.xml; Size: 33,498 bytes; and Date of Creation: Mar. 6, 2025) is herein incorporated by reference in its entirety.

### FIELD OF INVENTION

The present invention is in the field of in vivo phototagging.

### BACKGROUND OF THE INVENTION

Information processing in neural circuits requires precise interactions between molecularly and functionally diverse populations of neurons. Since gene expression ultimately dictates neuronal connectivity and function, a fundamental goal of neuroscience has been to characterize gene expression profiles of functionally defined neurons and to measure changes in gene expression associated with distinct functional states of neurons. High-throughput transcriptomic approaches such as single-cell/-nucleus RNA-seq (sc/snRNA-seq) have greatly accelerated the identification of gene programs in molecularly distinct types of neurons at single-cell resolution. However, subsequent in vivo and ex vivo functional and anatomical characterization of neuronal subtypes remains staggeringly slow as it requires the generation and validation of subtype-specific molecular tools. Recently, correlated in vivo Ca2+imaging with post hoc spatial transcriptomics has been used to relate gene expression with in vivo function, but this approach is limited to spatially sparse GABAergic interneurons. Therefore, a method to identify genes that are differently expressed in densely packed but functionally distinct glutamatergic pyramidal neurons (PNs), could significantly accelerate the understanding of how gene expression determines circuit function and behavior.

The inability to tag single functionally identified cortical PNs in vivo in behaving animals presents a significant challenge, as large-scale neural recordings have shown that PNs are highly heterogeneous in their physiological, anatomical, and response properties, and are spatially intermixed within neocortical and hippocampal circuits. For example, PNs with distinct spatial coding properties are distributed throughout the dense cell body layer of the hippocampus. However, the origin of this functional diversity in feature selectivity is largely unknown, and it remains unclear if gene expression differences are associated with discrete and transient functional cell states. Thus, there is a critical unmet need for function-forward approaches that directly map in vivo physiological and transcriptional profiles in cortical circuits in behaving animals.

Previous attempts using Ca2+ and light-dependent labeling of transiently active neurons (Moeyaert, B. et al. “Improved methods for marking active neuron populations”, Nat. Commun. 9, 4440 (2018)) were limited by their spatial resolution, deficiencies in targeting neurons with high baseline intracellular Ca2+ levels, and the inability to label neurons that decrease their activity in response to behavioral state or sensory stimuli. Similarly, immediate early gene-dependent labeling approaches lack the temporal and spatial resolution to faithfully report the precise activity patterns and response properties of single neurons. Finally, previous attempts to tag cortical neurons with photoactivatable fluorescent proteins at single-cell cellular precision have been deployed with limited success. A new method to directly map, in vivo, functional and transcriptional profiles in densely packed neurons is therefore greatly needed.

### SUMMARY OF THE INVENTION

The present invention provides nucleic acid molecules comprising at least one transcription regulatory element operably linked to an open reading frame, wherein the open reading frame encodes an RNA transcript encoding GCaMP7f, a ribosomal skipping peptide, and a fusion protein of a nuclear protein and photoactivatable red fluorescent protein are provided. Expression vectors and cells comprising the nucleic acid molecules are also provided, as are methods of using the nucleic acid molecules for simultaneous labeling and measuring calcium and analyzing a target cell.

According to a first aspect, there is provided a nucleic acid molecule comprising at least one transcription regulatory element operably linked to an open reading frame, wherein the open reading frame encodes a single RNA transcript encoding GCaMP7f, a ribosomal skipping peptide, and a fusion protein of a nuclear protein and photoactivatable red fluorescent protein.

According to some embodiments, the GCaMP7f comprises the amino acid sequence

(SEQ ID NO: 2)
MGSHHHHHHGMASMTGGQQMGRDLYDDDDKDLATMVDSSRRKWNKTGHA
VRAIGRLSSLENVYIKADKQKNGIKANFKIRHNIEDGGVQLAYHYQQNT
PIGDGPVLLPDNHYLSVQSKLSKDPNEKRDHMVLLEFVTAAGITLGMDE
LYKGGTGGSMVSKGEELFTGVVPILVELDGDVNGHKFSVSGEGEGDATY
GKLTLKFICTTGKLPVPWPTLVTTLTYGVQCFSRYPDHMKQHDFFKSAM
PEGYIQERTIFFKDDGNYKTRAEVKFEGDTLVNRIELKGIDFKEDGNIL
GHKLEYNLPDQLTEEQIAEFKELFSLFDKDGDGTITTKELGTVMRSLGQ
NPTEAELQDMINEVDADGDGTIDFPEFLTMMARKMKYTDSEEEIREAFR
VFDKDGNGYISAAELRHVMTNLGEKLTDEEVDEMIREADIDGDGQVNYE
EFVQMMTAK.

According to some embodiments, the ribosomal skipping peptide is selected from P2A, T2A, E2A and F2A.

According to some embodiments, the ribosomal skipping peptide is P2A comprising the amino acid sequence ATNFSLLKQAGDVEENPGP (SEQ ID NO: 8).

According to some embodiments, the nucleic acid molecule comprises a sequence encoding GSG directly 5′ to a sequence encoding the ribosomal skipping peptide.

According to some embodiments, the photoactivatable red fluorescent protein is a PamCherry protein.

According to some embodiments, the PAmCherry protein is PamCherryl and comprises the amino acid sequence:

(SEQ ID NO: 4)
MVSKGEEDNMAIIKEFMRFKVHMEGSVNGHVFEIEGEGEGRPYEGTQTA
KLKVTKGGPLPFTWDILSPQFMYGSNAYVKHPADIPDYFKLSFPEGFKW
ERVMKFEDGGVVTVTQDSSLQDGEFIYKVKLRGTNFPSDGPVMQKKTMG
WEALSERMYPEDGALKGEVKPRVKLKDGGHYDAEVKTTYKAKKPVQLPG
AYNVNRKLDITSHNEDYTIVEQYERAEGRHSTGGMDELYK.

According to some embodiments, the nuclear protein is a histone.

According to some embodiments, the histone is Histone 2B (H2B).

According to some embodiments, the H2B is H2B type 1-J and comprises the amino acid sequence

(SEQ ID NO: 16)
MPEPAKSAPAPKKGSKKAVTKAQKKGGKKRKRSRKESYSIYVYKVLKQV
HPDTGISSKAMGIMNSFVNDIFERIAGEASRLAHYNKRSTITSREIQTA
VRLLLPGELAKHAVSEGTKAITKYTSAK

According to some embodiments, the fusion protein comprises a peptide linker between the histone and the photoactivatable red fluorescent protein, and wherein the peptide linker is between 1 and 10 amino acids is length.

According to some embodiments, the nucleic acid molecule further comprises a linker sequence encoding an amino acid linker between the ribosomal skipping peptide and the fusion protein, wherein the amino acid linker is 4-10 amino acids in length.

According to some embodiments, the amino acid linker comprises four consecutive alanine residues.

According to some embodiments, the single RNA transcript encodes SEQ ID NO: 22.

According to some embodiments, the open reading frame comprises SEQ ID NO: 21.

According to some embodiments, the transcription regulatory element is a promoter.

According to some embodiments, the promoter is active in cortical glutamatergic neurons.

According to some embodiments, the promoter is the CAMKII promoter or a fragment thereof that drives transcription in cortical glutamatergic neurons.

According to some embodiments, the CAMKII promoter or fragment thereof comprises

(SEQ ID NO: 23)
cacttgtggactaagtttgttcgcatccccttctccaaccccctcagta
catcaccctgggggaacagggtccacttgctcctgggcccacacagtcc
tgcagtattgtgtatataaggccagggcaaagaggagcaggttttaaag
tgaaaggcaggcaggtgttggggaggcagttaccggggcaacgggaaca
gggcgtttcggaggtggttgccatggggacctggatgctgacgaaggct
cgcgaggctgtgagcagccacagtgccctgctcagaagccccaagctcg
tcagtcaagccggttctccgtttgcactcaggagcacgggcaggcgagt
ggcccctagttctgggggcagc.

According to another aspect, there is provided an expression vector comprising the nucleic acid molecule of the invention.

According to some embodiments, the expression vector is an adeno-associated viral vector (AAV).

According to another aspect, there is provided a cell comprising a nucleic acid molecule of the invention.

According to another aspect, there is provided a method of simultaneously fluorescently labeling and measuring calcium in a target cell, the method comprising expressing a nucleic acid molecule of the invention in the target cell, thereby simultaneously fluorescently labeling and measuring calcium in a target cell.

According to some embodiments, the at least one transcription regulatory element is active in the target cell.

According to some embodiments, the target cell is a cortical glutamatergic neuron.

According to some embodiments, the method is an in vivo method.

According to some embodiments, the method further comprises shinning on the target cell an 810-840 nm excitation light or an equivalent light that photoconverts the photoactivatable red fluorescent protein, a 920-960 nm excitation light or an equivalent light that excites the GCaMP7f, and a 1040 nm excitation light or an equivalent light that excites the photoactivated photoactivatable red fluorescent protein.

According to another aspect, there is provided a method of analyzing a target functional active cell, the method comprising,
a. receiving a mixture of activated cells and non-activated cells or a tissue comprising activated cells expressing a nucleic acid molecule of the invention and comprising green fluorescence detected at 474-575 nm;
b. isolating a cell comprising red fluorescence detected at between 600-630 nm; and
c. performing RNA-sequencing (RNA-seq) on RNA from the isolated cell; thereby analyzing a target functionally active cell.

According to some embodiments, a single cell is isolated and the RNA-seq is single cell RNA-seq.

According to some embodiments, 20-200 cells are isolated and the RNA-seq is mesoscale RNA sequencing (Meso-seq).

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1J: In vivo two-photon phototagging with 2P-NucTag. (1A) Schematics of the 2P-NucTag pipeline. Top: bicistronic rAAV construct, injection to the hippocampus. Middle: in vivo two-photon (2P) GCaMP-Ca2+ population imaging followed by 2P PAmCherry photoactivation, fluorescence-activated cell sorting (FACS), and mesoscale sequencing (Meso-seq). (1B) Top: representative in vivo time-averaged (6 frames average) 2P images of individual cells before (Pre) and after (Post) in vivo two-photon PAmCherry photoactivation in the CA1 pyramidal layer of the mouse dorsal hippocampus. Individual nuclei were photoactivated with 810-nm 2P laser chessboard scanning region-of-interest (ROI, yellow boxes) over target nuclei (70×70 pixel for each ROI, 0.1 μm/px, 1.3 ms/px total pixel dwell time, 6,370 ms total scan time per ROI. Laser power was 40 mW measured after the objective) with a 3-dimensional acousto-optical deflector microscope (3D-AOD). Gray: GCaMP7f (940 nm excitation), magenta: PAmCherry, (1040 nm excitation). Scale bar: 50 μm. Middle and bottom: imprints of letters ‘BI’ and ‘ZI’ following patterned in vivo two-photon photoactivation in the hippocampal CA1 pyramidal layer (scale bar, 50 μm). (1C) Characterization of in vivo 2P photoactivation parameters for PAmCherry: duration, wavelength, laser power (measured after the objective) (n=11-12 cells per condition). Relative change in PAmCherry red fluorescence (AF) is based on normalizing the tagged nuclei fluorescence to the fluorescence of neighboring untagged nuclei measured with 1040 nm excitation. (1D) In vivo stability of the PAmCherry fluorescence signal over days after a single photoactivation scan (n=8 cells). (1E) Representative time-averaged images from z-stacks of photoactivated nuclei in vivo (magenta: PAmCherry, scale bar: 100 μm). Middle: ex vivo post hoc confocal z-stack image of the same field of view (FOV, magenta: PAmCherry). Right: registered in vivo and ex vivo images following non-rigid image transformation (magenta: in vivo, yellow: ex vivo, see methods). (1F) Left: 3D overlay of tagged nuclei registered between in vivo (magenta) and ex vivo (yellow) z-stacks with increasing lateral resolution (as in 1E). Gray box represents the segmented area for subsequent images. Right: normalized lateral (x-y, left) and axial (z, right) fluorescence profiles (mean±s.e.m.) of tagged cells in vivo (magenta, n=1 mouse, 200 cells). Yellow: mean±s.e.m. of ex vivo confocal images (as in 1E and 1F, same mouse and nuclei). Inset: (x-y) top: average in vivo maximum z-projection, bottom: average ex vivo maximum z projection; (z) top: average in vivo lateral projection, bottom: average ex vivo lateral projection. Scale bar: 10 μm. Boxplots show the 25th, 50th (median), and 75th quartile ranges, with the whiskers extending to 1.5 interquartile ranges below or above the 25th or 75th quartiles, respectively. Outliers are defined as values extending beyond the whisker ranges. (1G) Representative average GCaMP-Ca2+ transients from nine CA1 PNs, vertical scale bar (50% AF/F). (1H) Left: GCaMP amplitude (54.6%+0.3% AF/F, n=8190 cells in 9 mice). Middle: GCaMP half rise time (0.15s+0.001s, n=7797 cells in 9 mice), Right: GCaMP half decay time (0.44s+0.003s, n=8282 cells in 9 mice, 940 nm excitation, See methods for cell exclusion criterion). Boxplots show the 25th, 50th (median), and 75th quartile ranges, with the whiskers extending to 1.5 interquartile ranges below or above the 25th or 75th quartiles, respectively. Outliers are defined as values extending beyond the whisker ranges. (11) Left: in vivo 3D visualization of the entire field of view (FOV). Middle: subset of in vivo 3D visualization. Right: representative cell from in vivo 3D visualization. (1J) Left: ex vivo confocal 3D visualization of entire FOV. Middle: subset of ex vivo 3D visualization. Right: representative cell from ex vivo 3D visualization. FIGS. 1I and 1J correspond to FIG. 1F.

FIGS. 2A-2N: Selective phototagging of place cells in the hippocampus with 2P-NucTag. (2A) Pipeline for two-photon (2P) phototagging of functionally identified hippocampal neurons during spatial navigation. (2B) Left: schematics of 2P imaging setup in virtual reality (VR). Head-fixed mice are trained to run for a water reward in a 4-m long linear VR corridor projected onto LCD screens surrounding the animal. At the end of the corridor, mice are teleported back to the start position after a 2-second delay. Right: example 2P field of view (FOV) of GCaMP in the CA1 pyramidal layer. Scale bar: 100 μm. (2C) Left: Traces of relative GCaMP-Ca2+ fluorescence changes (11F/F) from five example CA1 place cells during VR spatial navigation. Right: heatmaps of normalized 11F/F activity from three example place cells over 20 laps during VR navigation. (2D) Left: heatmap of all CA1PNs detected with Suite2p/Cellpose in the FOV shown in 2B. Identified place cells are marked with an orange box. Right: Zoomed-in heatmap of place cell tuning curves. (2E) Left: spatial mask (orange) of identified place cells from 2D in the FOV. Right: PAmCherry fluorescence (magenta) of tagged nuclei after 2P phototagging. Scale bar: 100 μm. (2F) Left: overlay of spatial masks of identified CAIPNs and tagged nuclei for the FOVs in E. Scale bar: 100 μm. Right: tagging efficacy, defined as the fraction of successfully tagged place cell nuclei (93.3%+4.2%, mean±s.e.m., n=5 mice). (2G) Left: Proportion of single, double, and triple-tagged nuclei following phototagging of a single place cell. Right: relative change in PAmCherry red fluorescence (1070 nm excitation) for non-tagged cells in the FOV after 2P imaging (green), after 2P phototagging of targeted place cell nuclei (orange) and off-target nuclei (gray, n=5 mice). Boxplots show the 25th, 50th (median), and 75th quartile ranges, with the whiskers extending to 1.5 interquartile ranges below or above the 25th or 75th quartiles, respectively. Outliers are defined as values extending beyond the whisker ranges. (2H) Spatial mask (blue) of identified ‘silent’ cells from the FOV in B. Scale bar: 100 μm. (21) Left: PAmCherry fluorescence (magenta) of tagged nuclei after 2P phototagging. Right: overlay of spatial masks of identified CAIPNs and tagged nuclei for the FOVs in 2D and 2E. Note only a subset of silent cells present in the FOV were tagged. Scale bar: 100 μm. (2J) Left: Proportion of single, double, and triple-tagged nuclei following phototagging of a single silent cell. Right: relative PAmCherry fluorescence change for non-tagged cells in the FOV after 2P imaging (green), after 2P phototagging of targeted silent cell nuclei (orange) and off-target nuclei (gray, n=4 mice). (2K) Average velocity of the mice during virtual reality navigation task (Mann-Whitney U test, p-value=0.286). (2L) Left: deconvolved events per minute from all cells across all mice from 2P GCaMP-Ca2+imaging (averaged across mice, n=5 ‘Place’ mice, n=4 ‘Silent’ mice, Mann-Whitney U Test, p-value=0.28). Right: deconvolved events per minute from place cells across all mice (averaged across mice, place n=5, silent n=4, Mann-Whitney U Test, p-value=0.90). (2M) Left: GCaMP transient amplitude of all cells between groups (averaged across mice, n=5 ‘Place’ mice, n=4 ‘Silent’ mice, Mann-Whitney U Test, p-value=0.14), Middle: GCaMP half rise time of all cells between groups (averaged across mice, n=5 ‘Place’ mice, n=4 ‘Silent’ mice, Mann-Whitney U Test, p-value=0.14), Right: GCaMP half decay time of all cells between groups (averaged across mice, n=5 ‘Place’ mice, n=4 ‘Silent’ mice, Mann-Whitney U Test, p-value=0.81). Boxplots show the 25th, 50th (median), and 75th quartile ranges, with the whiskers extending to 1.5 interquartile ranges below or above the 25th or 75th quartiles, respectively. Outliers are defined as values extending beyond the whisker ranges. (2N) From left to right by column: animal ID (animals in FIG. 2A-2M are not shown here), in vivo two-photon (2P) imaging fields of view (FOVs), functionally defined masks (orange for place cells and blue for silent cells), PAmCherry fluorescence (magenta) of tagged nuclei after 2P phototagging and overlay of spatial masks from identified CAIPNs and tagged nuclei for the respective FOV in the same row. Note that only a subset of silent cells present in the FOV in ‘Silent’ mice were tagged, in order to approximate the number of phototagged place cells in ‘Place’ mice. Scale bar: 100 μm.

FIGS. 3A-3H: Post hoc transcriptional profiling of phototagged place and silent cells. (3A) Schematics of in vivo photoactivated nuclei. ‘Place’ cell sample and ‘Silent’ cell samples from different mice were collected for FACS and Meso-seq. (3B) Representative FACS graph. Gating for mCherry was set after the first 5000 events of DAPI+ nuclei to the border of the ‘dim’ mCherry+ population to separate out the sparse and high-intensity mCherry+ population. Bright mCherry+ NeuN+ populations were collected as the photoactivated nuclei. (3C) Top: number of FACS sorted nuclei from ‘place’ and ‘silent’ samples (n=9, 17-79 sorted nuclei, 40.3+6.43, mean±s.e.m.). Bottom: Proportion of FACS sorted nuclei compared to the number of in vivo photoactivated nuclei (n=9, 18.45% to 66.39% FACS recovery, 39.94+6.30%, mean±s.e.m.). (3D) Volcano plot of Meso-seq differential expressed gene (DEG) analysis for ‘place’ and ‘silent’ cells (significantly different genes are shown in orange and blue. Orange: enriched in place cells; blue: enriched in silent cells). (3E) Meso-seq MA plot depicting DeSeq2 normalized gene counts versus log 2 fold change of silent/place samples. Genes that are significantly different are labeled in orange and blue (same as above). Genes shown in 3F & 3G are highlighted and labeled in 3E. (3F) Bar graph showing the normalized counts for genes that are not differentially expressed (FDR adjusted p-value. *<0.05, **<0.001, ***<0.001, PyDeSeq2. Otherwise, comparisons are not significant). (3G) Bar graph showing the normalized counts for differentially expressed genes (FDR adjusted p-value. *<0.05, **<0.001, ***<0.001, PyDeSeq2. Otherwise, comparisons are not significant). (H) Gene ontology analysis performed on all differentially expressed genes. Vertical line: FDR-adjusted p value of 0.05. NES=normalized enrichment score. Boxplots show the 25th, 50th (median), and 75th quartile ranges, with the whiskers extending to 1.5 interquartile ranges below or above the 25th or 75th quartiles, respectively. Outliers are defined as values extending beyond the whisker ranges.

FIGS. 4A-4B: Additional data on transcriptomics analysis of place and silent cells. (4A) Sequencing statistics for all ‘place’ and ‘silent’ cell samples. Total number of reads-40 to 60 million reads, 54.64+2.09, n=9. Percent mapped by STAR-80.58 to 86.92%. 84.33+0.73. Number of unique genes-14176 to 20990, 17486+714. (4B) Normalized counts for groups of genes plotted for ‘place’ versus ‘silent’. Here we show that gene expression of apoptotic genes, superficial CA1 genes, deep CA1 genes, housekeeping genes, proximal CA1 genes, and distal CA1 genes are not different between the two groups (FDR adjusted p-value. *<0.05, **<0.001, ***<0.001, PyDeSeq2. All comparisons in this figure are not significant).

FIGS. 5A-5D: Additional data on transcriptomics analysis of place and silent cells. (5A) Top: MA plot of ‘place’ versus ‘random’. Differentially expressed genes (DEGs) are labeled orange. Bottom: MA plot of ‘silent’ versus ‘random’. DEGs are labeled in blue. Both: DEGs that are common for ‘place’ versus ‘random’ were highlighted and labeled. (5B) Normalized counts for 4 example genes that are significantly differentially expressed across comparisons (FDR adjusted p-value. *<0.05, **<0.001, ***<0.001, PyDeSeq2. Showing here a comparison of ‘place’ versus ‘random’ or ‘silent’ versus ‘random’. ‘Place’ versus ‘silent’ comparisons were shown in FIG. 3). (5C) MA plot of male versus female for the ‘random’ dataset. Top: Y-linked genes that are differentially expressed between sex are highlighted and labeled. Bottom: same for genes in 5A and 5B are highlighted and labeled. They are not differentially expressed between sex (FDR adjusted p-value. *<0.05, **<0.001, ***<0.001, PyDeSeq2. Otherwise, comparisons are not significant). (5D) Normalized counts for 3 example DEGs between male and female.

FIG. 6: Post hoc immunohistochemistry and in situ hybridization on tagged tissue. Top and middle: confocal horizontal images of post hoc calbindin immunohistochemistry on tissue with tagged cells in the hippocampus. Scale bar: 50 μm (top), 20 μm (middle, higher magnification). Bottom: confocal horizontal images of tagged tissue hybridized with an RNAScope probe for Calb1, highlighting tagged cells with positive Calb1 signal. Scale bar: 20 μm.

### DETAILED DESCRIPTION OF THE INVENTION

The present invention, in some embodiments, provides nucleic acid molecules comprising at least one transcription regulatory element operably linked to an open reading frame, wherein the open reading frame encodes a single RNA transcript encoding GCaMP7f, a ribosomal skipping peptide, and a fusion protein of a nuclear protein and photoactivatable red fluorescent protein are provided. Expression vectors and cells comprising the nucleic acid molecules are also provided, as are methods of using the nucleic acid molecules for simultaneous labeling and measuring calcium and analyzing a target cell.

The invention is based, at least in part, on the creation of a robust in vivo pipeline (2P-NucTag), based on a photoactivatable red fluorescent protein (PAmCherry) and a genetically encoded green Ca2+ indicator (GCaMP7f), that optimizes a previously described framework (Lee, et al., “Sensory coding mechanisms revealed by optical tagging of physiologically defined neuronal types”, Science 366, 1384-1389 (2019), the contents of which are hereby incorporated by reference in their entirety) mainly used ex vivo. The GCaMP7f protein was found to be surprisingly superior to other indicators used in the past. The instant approach combines large-scale in vivo two-photon (2P) functional imaging of cortical PNs with reliable and selective 2P phototagging of nuclei in a subset of neurons based on their functional properties. Using fluorescence-activated cell sorting (FACS) to isolate phototagged neuronal nuclei post hoc, combined with the recently developed Meso-seq approach for transcriptomics in ultra-sparse populations (Apelblat, et al., “Meso-seq for in-depth transcriptomics in ultra-low amounts of FACS-purified neuronal nuclei”, Cell Rep Methods 2, 100259 (2022), the contents of which are hereby incorporated by reference in its entirety), previously unattainable molecular characterization of functionally identified PNs in vivo in behaving animals was achieved.

By a first aspect, there is provided a nucleic acid molecule comprising an open reading frame encoding GCaMP7f and a red fluorescent protein.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C).

The terms “nucleic acid molecule” include but not limited to single-stranded RNA (ssRNA), double-stranded RNA (dsRNA), single-stranded DNA (ssDNA), double-stranded DNA (dsDNA), small RNA such as miRNA, siRNA and other short interfering nucleic acids, snoRNAs, snRNAs, tRNA, piRNA, tnRNA, small rRNA, hnRNA, lncRNA, circulating nucleic acids, fragments of genomic DNA or RNA, degraded nucleic acids, ribozymes, viral RNA or DNA, nucleic acids of infectious origin, amplification products, modified nucleic acids, plasmidical or organellar nucleic acids and artificial nucleic acids such as oligonucleotides. In some embodiments, the nucleic acid molecule is a DNA. In some embodiments, the nucleic acid molecule is an RNA.

In some embodiments, the nucleic acid molecule is a vector. In some embodiments, the vector is a DNA vector. In some embodiments, the vector is a plasmid. In some embodiments, the vector is an expression vector. In some embodiments, the vector is a viral vector. In some embodiments, the viral vector is an adenoviral vector. In some embodiments, the viral vector is an adeno-associated viral vector (AAV).

A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

The vector may be a DNA plasmid delivered via non-viral methods or via viral methods. The viral vector may be a retroviral vector, a herpesviral vector, an adenoviral vector, an adeno-associated viral vector or a poxviral vector. In some embodiments, the vector is introduced into the cell by standard methods including electroporation (e.g., as described in From et al., Proc. Natl. Acad. Sci. USA 82, 5824 (1985)),Heat shock, infection by viral vectors, high velocity ballistic penetration by small particles with the nucleic acid either within the matrix of small beads or particles, or on the surface (Klein et al., Nature 327. 70-73 (1987)), and/or the like.

In some embodiments, the nucleic acid molecule comprises at least one transcription regulatory element. In some embodiments, the at least one transcription regulatory element is operably linked to the open reading frame. The term “operably linked” is intended to mean that the nucleotide sequence of interest (i.e., the open reading frame) is linked to the regulatory element or elements in a manner that allows for expression of the nucleotide sequence (e.g. in an in vitro transcription/translation system or in a host cell when the vector is introduced into the host cell).

In some embodiments, the at least one transcription regulatory element is a promoter. The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins. In some embodiments, the promoter is a constitutive promoter. In some embodiments, the promoter is an inducible promoter. In some embodiments, the promoter is a tissue specific promoter. In some embodiments, the promoter is a cell or cell type specific promoter. In some embodiments, the cell is the target cell. In some embodiments, the promoter is active in the target cell. In some embodiments, target cell is a neuron. In some embodiments, the neuron is a glutamatergic neuron. In some embodiments, the neuron is a cortical neuron. In some embodiments, the neuron is a cortical glutamatergic neuron.

In some embodiments, the promoter is the Ca 2+/calmodulin-dependent protein kinase II (CaMKII) promoter or a fragment thereof. In some embodiments, CAMKII is human CAMKII. In some embodiments, the fragment is a fragment that drives transcription. In some embodiments, driving transcription is driving transcription in the target cell. In some embodiments, driving transcription is driving transcription in cortical glutamatergic neurons. In some embodiments, the CAMKII promoter or fragment thereof comprises the nucleotide sequence cacttgtggactaagtttgttcgcatccccttctccaaccccctcagtacatcaccctgggggaacagggtccacttgctcctgggcc cacacagtcctgcagtattgtgtatataaggccagggcaaagaggagcaggttttaaagtgaaaggcaggcaggtgttggggag gcagttaccggggcaacgggaacagggcgtttcggaggtggttgccatggggacctggatgctgacgaaggctcgcgaggctg tgagcagccacagtgccctgctcagaagccccaagctcgtcagtcaagccggttctccgtttgcactcaggagcacgggcaggc gagtggcccctagttctgggggcagc (SEQ ID NO: 23). In some embodiments, the sequence of the CAMKII promoter or fragment thereof consists of SEQ ID NO: 23.

In some embodiments, nucleic acid sequences are transcribed by RNA polymerase II (RNAP II and Pol II). RNAP II is an enzyme found in eukaryotic cells. It catalyzes the transcription of DNA to synthesize precursors of mRNA and most snRNA and microRNA.

In some embodiments, mammalian expression vectors include, but are not limited to, pcDNA3, pcDNA3.1 (+), pGL3, pZeoSV2 (+), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p205. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as lateral infection and targeting specificity, are used for in vivo expression. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells. In some embodiments, the vector is an AAV. In some embodiments, the AAV is a recombinant AAV (rAAV). Examples of AAV serotypes that may be used include, for example, AAV1, AAV2, AAV3, AAV4, AAV5, AAV6, AAV6.2, AAV7, AAV8, AAV9, AAVrh10, AAVDJ, AAVDJ/8, AAVPHP.eB, AAVPHP.S, AAV2-retro, AAV2-QuadYF, and AAV2.7m8. In some embodiments, the AAV is AAVDJ.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

It will be appreciated that other than containing the necessary elements for the transcription and translation of the inserted coding sequence (encoding the polypeptide), the expression construct of the present invention can also include sequences engineered to optimize stability, production, purification, yield or activity of the expressed polypeptide.

A person with skill in the art will appreciate that a gene can also be expressed from a nucleic acid construct administered to the individual employing any suitable mode of administration, described herein (i.e., in vivo phototagging). In one embodiment, the nucleic acid construct is administered to a subject. In one embodiment, the nucleic acid construct is administered to a subject and the AAV has tropism to the target cell. As used herein, the terms “administering,” “administration,” and like terms refer to any method which, in sound medical practice, delivers a composition containing a molecule of the invention to a subject in such a manner as to provide a phototagging effect. In some embodiments, the administering is systemic administering. In some embodiments, the administering is local administering. Suitable routes of administration include parenteral, subcutaneous, intravenous, intramuscular, oral, intranasal, intraventricular, intraparenchymal, or intraperitoneal.

The dosage administered will be dependent upon the age, health, and weight of the recipient, kind of concurrent treatment, if any, frequency of treatment, and the nature of the effect desired.

In some embodiments, the open reading frame is an RNA. In some embodiments, the open reading frame encodes an RNA. In some embodiments, an RNA is an RNA transcript. In some embodiments, the RNA transcript is a single RNA transcript. It will be understood by the skilled artisan that rather than having two separate open reading frames, one of the GCaMP7f and one for the red fluorescent protein, the instant invention makes use of a single open reading frame but with a linker that allows for separation of the two proteins. This results in the production of two separate proteins, but ensures that any cell that receives the first protein also receives the second and that the two proteins are produced in the exact same amounts.

GCaMP is a calcium indicator that can be used to measure Ca2+ levels in cells and thereby their activity (e.g. neuronal activity). It is a synthetic fusion of green fluorescent protein (GFP), calmodulin (CaM), and M13, a peptide sequence from myosin light-chain kinase. When bound to Ca2+, GCaMP fluoresces green with a peak excitation wavelength of 480 nm and a peak emission wavelength of 510 nm. GCaMP7f was first disclosed in Dana et al., 2019, “High-performance calcium sensors for imaging activity in neuronal populations and microcompartments”, Nature Methods, Jul; 16 (7): 649-657, the contents of which are hereby incorporated by reference in its entirety.

In some embodiments, GCaMP7f is jGCaMP7f. In some embodiments, GCaMP7f comprises the amino acid sequence MGSHHHHHHGMASMTGGQQMGRDLYDDDDKDLATMVDSSRRKWNKTGHAVR AIGRLSSLENVYIKADKQKNGIKANFKIRHNIEDGGVQLAYHYQQNTPIGDGPVLL PDNHYLSVQSKLSKDPNEKRDHMVLLEFVTAAGITLGMDELYKGGTGGSMVSKG EELFTGVVPILVELDGDVNGHKFSVSGEGEGDATYGKLTLKFICTTGKLPVPWPTL VTTLTYGVQCFSRYPDHMKQHDFFKSAMPEGYIQERTIFFKDDGNYKTRAEVKFE GDTLVNRIELKGIDFKEDGNILGHKLEYNLPDQLTEEQIAEFKELFSLFDKDGDGTI TTKELGTVMRSLGQNPTEAELQDMINEVDADGDGTIDFPEFLTMMARKMKYTDS EEEIREAFRVFDKDGNGYISAAELRHVMTNLGEKLTDEEVDEMIREADIDGDGQV NYEEFVQMMTAK (SEQ ID NO: 2). In some embodiments, the GCaMP7f amino acid sequence consists of SEQ ID NO: 2. In some embodiments, GCaMP7f is encoded by a nucleotide sequence comprising SEQ ID NO: 1. In some embodiments, GCaMP7f is encoded by a nucleotide sequence consisting of SEQ ID NO: 1. In some embodiments, the open reading frame comprises SEQ ID NO: 1, which encodes GCaMP7f.

In some embodiments, the sequence encoding the GCaMP7F is separated from the sequence encoding the red fluorescent protein by a linker. In some embodiments, the GCaMP7F is separated from the red fluorescent protein by a linker. As used herein, the term “linker” can refer to the protein linker itself or the nucleotide sequence that encodes the protein linker. In some embodiments, the linker is a cleavable linker. In some embodiments, the linker is a self-cleaving linker. In some embodiments, the linker is a peptide linker. In some embodiments, the linker is a ribosomal skipping peptide. In some embodiments, the open reading frame encodes GCaMP7f, a ribosomal skipping peptide, and a red fluorescent protein.

In some embodiments, the linker comprises a 2A self-cleaving peptide. In some embodiments, the linker consists of a 2A self-cleaving peptide. In some embodiments, the self-cleaving linker induces ribosome skipping. In some embodiments, the self-cleaving linker induces failure to make a peptide bond between an amino acid and the next amino acid to be produced by the open reading frame thus resulting in two separate proteins being produced. As used herein, a “2A self-cleaving peptide” is the same as a “2A ribosomal skipping peptide”. In some embodiments, the 2A peptide comprises the motif DX1EX2NPGP (SEQ ID NO: 12) wherein X1 is any amino acid and X2 is any amino acid. In some embodiments, SEQ ID NO: 12 is DVEXNPGP (SEQ ID NO: 13), wherein X is E or S. In some embodiments, the 2A peptide is selected from a T2A peptide, a P2A peptide, an E2A peptide and an F2A peptide.

In some embodiments, the 2A peptide is a P2A peptide. In some embodiments, the linker comprises a P2A peptide. In some embodiments, the linker consists of a P2A peptide. In some embodiments, the ribosomal skipping peptide comprises a P2A peptide. In some embodiments, the ribosomal skipping peptide consists of a P2A peptide. In some embodiments, the P2A is encoded by peptide the sequence gccacgaacttctctctgttaaagcaagcaggagacgtggaagaaaaccccggtcct (SEQ ID NO: 7). In some embodiments, the linker is encoded by a sequence comprising SEQ ID NO: 7. In some embodiments, the linker is encoded by a sequence consisting of SEQ ID NO: 7. In some embodiments, the ribosomal skipping peptide is encoded by a sequence comprising SEQ ID NO: 7. In some embodiments, the ribosomal skipping peptide is encoded by a sequence consisting of SEQ ID NO: 7. In some embodiments, the P2A peptide comprises the amino acid sequence ATNFSLLKQAGDVEENPGP (SEQ ID NO: 8). In some embodiments, the P2A peptide consists of the amino acid sequence of SEQ ID NO: 8. In some embodiments, the linker comprises or consists of SEQ ID NO: 8. In some embodiments, the ribosomal skipping peptide comprises or consists of SEQ ID NO: 8.

In some embodiments, the 2A peptide is a T2A peptide. In some embodiments, the linker comprises a T2A peptide. In some embodiments, the linker consists of a T2A peptide. In some embodiments, the ribosomal skipping peptide comprises a T2A peptide. In some embodiments, the ribosomal skipping peptide consists of a T2A peptide. In some embodiments, the T2A peptide comprises the amino acid sequence

EGRGSLLTCGDVEENPGP (SEQ ID NO: 9). In some embodiments, the T2A peptide consists of the amino acid sequence of SEQ ID NO: 9. In some embodiments, the linker comprises or consists of SEQ ID NO: 9. In some embodiments, the ribosomal skipping peptide comprises or consists of SEQ ID NO: 9.

In some embodiments, the 2A peptide is an E2A peptide. In some embodiments, the linker comprises an E2A peptide. In some embodiments, the linker consists of an E2A peptide. In some embodiments, the ribosomal skipping peptide comprises an E2A peptide. In some embodiments, the ribosomal skipping peptide consists of an E2A peptide. In some embodiments, the E2A peptide comprises the amino acid sequence QCTNYALLKLAGDVESNPGP (SEQ ID NO: 10). In some embodiments, the E2A peptide consists of the amino acid sequence of SEQ ID NO: 10. In some embodiments, the linker comprises or consists of SEQ ID NO: 10. In some embodiments, the ribosomal skipping peptide comprises or consists of SEQ ID NO: 10.

In some embodiments, the 2A peptide is an F2A peptide. In some embodiments, the linker comprises an F2A peptide. In some embodiments, the linker consists of an F2A peptide. In some embodiments, the ribosomal skipping peptide comprises an F2A peptide. In some embodiments, the ribosomal skipping peptide consists of an F2A peptide. In some embodiments, the F2A peptide comprises the amino acid sequence VKQTLNFDLLKLAGDVESNPGP (SEQ ID NO: 11). In some embodiments, the F2A peptide consists of the amino acid sequence of SEQ ID NO: 11. In some embodiments, the linker comprises or consists of SEQ ID NO: 11. In some embodiments, the ribosomal skipping peptide comprises or consists of SEQ ID NO: 11.

In some embodiments, the ribosomal skipping peptide comprises an N-terminal GSG. In some embodiments, a sequence encoding GSG is directly 5′ to the sequence encoding the ribosomal skipping peptide. In some embodiments, the linker comprises GSG followed by the ribosomal skipping peptide. In some embodiments, the open reading frame comprises a sequence encoding GSG directly 5′ to a sequence encoding the ribosomal skipping peptide.

In some embodiments, the red fluorescent protein is a photoactivatable red fluorescent protein. A photoactivatable fluorescent protein is a fluorescent protein that display unique changes in its spectral properties upon exposure to a specific wavelength of light. In some embodiments, the photoactivatable red fluorescent protein is PA-mRFP. In some embodiments, the photoactivatable red fluorescent protein is a PAmCherry protein. In some embodiments, the PAmCherry protein is selected from PAmCherry1, PAmCherry2, and PAmCherry3. In some embodiments, the PAmCherry protein is PAmCherry 1. In some embodiments, PAmCherry1 is encoded by a nucleotide sequence comprising SEQ ID NO: 3. In some embodiments, PAmCherry1 is encoded by a nucleotide sequence consisting of SEQ ID NO: 3. In some embodiments, the open reading frame comprises SEQ ID NO: 3. In some embodiments, PAmCherry 1 comprises the amino acid sequence MVSKGEEDNMAIIKEFMRFKVHMEGSVNGHVFEIEGEGEGRPYEGTQTAKLKVT KGGPLPFTWDILSPQFMYGSNAYVKHPADIPDYFKLSFPEGFKWERVMKFEDGGV VTVTQDSSLQDGEFIYKVKLRGTNFPSDGPVMQKKTMGWEALSERMYPEDGALK GEVKPRVKLKDGGHYDAEVKTTYKAKKPVQLPGAYNVNRKLDITSHNEDYTIVE QYERAEGRHSTGGMDELYK (SEQ ID NO: 4). In some embodiments, PAmCherry1 consists of the amino acid sequence of SEQ ID NO: 4.

In some embodiments, the PAmCherry protein is PAmCherry2. In some embodiments, PAmCherry2 comprises the amino acid sequence MVSKGEEDNMAIIKEFMRFKVHLEGSVNGHEFEIEGEGEGRPYEGTQTAKLKVTK GGPLPFAWDILSPQFMYGSNAYVKHPADIPDYFKLSFPEGFKWERVMNFEDGGVV TVTQDSSLQDGEFIYKVKLRGTNFPSDGPVMQKKTMGWETLSERMYPEDGALKG ELKARTKLKDGGHYDTEVKTTYKAKKPVQLPGAYNVNRKLDITSHNEDYTIVEQ YERAEGLHSTGGMDELYK (SEQ ID NO: 5). In some embodiments, PAmCherry2 consists of the amino acid sequence of SEQ ID NO: 5. In some embodiments, the PAmCherry protein is PAmCherry3. In some embodiments, PAmCherry3 comprises the amino acid sequence MVSKGEEDNMAIIKEFMRFKVHLEGSVNGHEFEIEGEGEGRPYEGTQTAKLKVTK GGPLPFTWDILSPQFMYGSNAYVKHPADIPDYFKLSFPEGFKWERVMNFEDGGVV TVTQDSSLQDGEFIYKVKLRGTNFPSDGPVIQKKTMGWDALSERMYPEDGALKGE LKARLKLKDGGHYEAEVKTTYKAKKPVQLPGAYNVNRKLDITSHNEDYTIVEQY ERAEGRHSTGGMDELYK (SEQ ID NO: 6). In some embodiments, PAmCherry3 consists of the amino acid sequence of SEQ ID NO: 6. In some embodiments, the photoactivatable red fluorescent protein is selected from SEQ ID NO: 4-6.

In some embodiments, the red fluorescent protein is a fusion protein. In some embodiments, the open reading frame encodes a fusion protein comprising the red fluorescent protein. As used herein, the term “fusion protein” refers to a single amino acid chain comprising parts of at least two different proteins. In some embodiments, the fusion protein comprises a photoactivatable red fluorescent protein. In some embodiments, a fusion is a fusion of a fragment from the proteins.

In some embodiments, the fusion protein is a fusion of a nuclear protein and the red fluorescent protein. In some embodiments, the fusion protein comprises a nuclear protein and the red fluorescent protein. In some embodiments, the nuclear protein is N-terminal to the red fluorescent protein. In some embodiments, the nuclear protein is C-terminal to the red fluorescent protein. In some embodiments, a nuclear protein is a protein comprising a nuclear localization signal/sequence (NLS). NLSes are well known in the art and any protein with an NLS may be used in the fusion protein. The NLS from the nuclear protein will serve to target the fusion protein to the nucleus as well. In some embodiments, the nuclear protein is N-terminal to the red fluorescent protein and the NLS of the nuclear protein targets the fusion protein to the nucleus. In some embodiments, the NLS comprises the amino acids sequence KX1X2X3, wherein X1 is K or R, X2 is any amino acid and X3 is K or R.

In some embodiments, the nuclear protein is a mammalian protein. In some embodiments, the nuclear protein is a human protein. In some embodiments, the nuclear protein is a histone. In some embodiments, the histone is selected from H1, H2A, H2B, H3, and H4. In some embodiments, the histone is a canonical histone or a histone variant. In some embodiments, the histone is a canonical histone. In some embodiments, the histone is Histone H2B (H2B). Sixteen variants of H2B are known in humans and any of the 16 may be used as the nuclear protein. In some embodiments, H2B is selected from H2B type 1, H2B type 2 and H2B type 3. In some embodiments, H2B is H2B type 1. H2B type 1 histones include: H2B type 1-A, H2B type 1-B, H2B type 1-C, H2B type 1-D, H2B type 1-H, H2B type 1-J, H2B type 1-K, H2B type 1-L, H2B type 1-M, H2B type 1-N, and H2B type 1-O.

In some embodiments, H2B is H2B type 1-J. In some embodiments, H2B type 1-J is encoded by a nucleotide sequence comprising SEQ ID NO: 15. In some embodiments, H2B type 1-J is encoded by a nucleotide sequence consisting of SEQ ID NO: 15. In some embodiments, the open reading frame comprises SEQ ID NO: 15. In some embodiments, the nuclear protein is encoded by SEQ ID NO: 15. In some embodiments, the sequence encoding the fusion protein comprises SEQ ID NO: 15. In some embodiments, H2B type 1-J comprises the amino acid sequence MPEPAKSAPAPKKGSKKAVTKAQKKGGKKRKRSRKESYSIYVYKVLKQVHPDTG ISSKAMGIMNSFVNDIFERIAGEASRLAHYNKRSTITSREIQTAVRLLLPGELAKHA

VSEGTKAITKYTSAK (SEQ ID NO: 16). In some embodiments, H2B type 1-J consists of SEQ ID NO: 16. In some embodiments, H2B type 1-J comprises the amino acid sequence MPEPAKSAPAPKKGSKKAVTKAQKKDGKKRKRSRKESYSIYVYKVLKQVHPDTG ISSKAMGIMNSFVNDIFERIAGEASRLAHYNKRSTITSREIQTAVRLLLPGELAKH AVSEGTKAVTKYTSAK (SEQ ID NO: 17). In some embodiments, H2B type 1-J consists of SEQ ID NO: 17. In some embodiments, H2B type 1-J is selected from SEQ ID NO: 16 and 17. In some embodiments, the nuclear protein is selected from SEQ ID NO: 16 and 17. In some embodiments, the fusion protein comprises SEQ ID NO: 16 or 17. In some embodiments, H2B type 1-J comprises the amino acid sequence MPEPAKSAPAPKKGSKKAVTKAQKKX1GKKRKRSRKESYSIYVYKVLKQVHPDT GISSKAMGIMNSFVNDIFERIAGEASRLAHYNKRSTITSREIQTAVRLLLPGELAKH AVSEGTKAX2TKYTSAK (SEQ ID NO: 14), wherein X1 is G or D and X2 is I or V. In some embodiments, H2B type 1-J consists of SEQ ID NO: 14.

In some embodiments, the fusion protein comprises a linker between the nuclear protein and the red fluorescent protein. In some embodiments, the fusion protein comprises a linker between the ribosomal skipping peptide and the fusion protein. In some embodiments, the fusion protein comprises a linker between the GCaMP7f and the ribosomal skipping peptide. In some embodiments, the linker is a spacer. In some embodiments, the linker is a peptide linker. In some embodiments, the linker comprises at least 1, 2, 3, 4, 5, 6, 7, or 8 amino acids. Each possibility represents a separate embodiment of the invention. In some embodiments, the linker comprises at least 1 amino acid. In some embodiments, the linker comprises at least a plurality of amino acids. In some embodiments, the linker comprises at least 6 amino acids. In some embodiments, the linker comprises at most 6, 7, 8, 9, 10, 11, 12, 13, 14, 15, 16, 17, 18, 19 or 20 amino acids. Each possibility represents a separate embodiment of the invention. In some embodiments, the linker comprises at most 10 amino acids. In some embodiments, the linker comprises 1-10 amino acids. In some embodiments, the linker comprises 4-10 amino acids. In some embodiments, the linker comprises 6 to 10 amino acids. In some embodiments, the linker comprises 8 to 10 amino acids. In some embodiments, the linker is 6 amino acids. In some embodiments, the linker comprises 6 to 15 amino acids. In some embodiments, the linker comprises 8 to 15 amino acids. In some embodiments, the linker is 6 amino acids. In some embodiments, the linker comprises DPPVAT (SEQ ID NO: 18). In some embodiments, the linker consists of SEQ ID NO: 18. In some embodiments, the linker comprises 4 consecutive amino acids. In some embodiments, the linker comprises AAAA (SEQ ID NO: 19). In some embodiments, the linker comprises DAAAAIAT (SEQ ID NO: 20). In some embodiments, the linker consists of SEQ ID NO: 20. The addition of a linker/spacer between the P2A peptide and the fusion protein was surprisingly beneficial and ensured the production of the full fusion protein and its proper targeting to the nucleus.

In some embodiments, the fusion protein is encoded by a sequence comprising SEQ ID NO: 24. In some embodiments, the fusion protein is encoded by a sequence consisting of SEQ ID NO: 24. In some embodiments, the open reading frame comprises SEQ ID NO: 24. In some embodiments, the fusion protein comprises the amino acid sequence MPEPAKSAPAPKKGSKKAVTKAQKKGGKKRKRSRKESYSIYVYKVLKQVHPDTG ISSKAMGIMNSFVNDIFERIAGEASRLAHYNKRSTITSREIQTAVRLLLPGELAKHA VSEGTKAITKYTSAKDPPVATMVSKGEEDNMAIIKEFMRFKVHMEGSVNGHVFEI EGEGEGRPYEGTQTAKLKVTKGGPLPFTWDILSPQFMYGSNAYVKHPADIPDYFK LSFPEGFKWERVMKFEDGGVVTVTQDSSLQDGEFIYKVKLRGTNFPSDGPVMQK KTMGWEALSERMYPEDGALKGEVKPRVKLKDGGHYDAEVKTTYKAKKPVQLPG AYNVNRKLDITS HNEDYTIVEQYERAEGRHSTGGMDELYK (SEQ ID NO: 25). In some embodiments, the amino acid sequence of the fusion protein consists of SEQ ID NO: 25.

In some embodiments, the open reading frame comprises SEQ ID NO: 21. In some embodiments, the open reading frame consists of SEQ ID NO: 21. In some embodiments, the RNA transcript encodes SEQ ID NO: 22. In some embodiments, the open reading frame encodes SEQ ID NO: 22. In some embodiments, the open reading frame encodes an amino acid sequence comprising SEQ ID NO: 22. In some embodiments, the open reading frame encodes an amino acid sequence consisting of SEQ ID NO: 22. In some embodiments, the RNA transcript encodes an amino acid sequence comprising SEQ ID NO: 22. In some embodiments, the RNA transcript encodes an amino acid sequence consisting of SEQ ID NO: 22.

By another aspect, there is provided an expression vector comprising a nucleic acid molecule of the invention.

By another aspect, there is provided a cell comprising a nucleic acid molecule of the invention.

By another aspect, there is provided a cell comprising an expression vector of the invention.

In some embodiments, the cell expresses the nucleic acid molecule. In some embodiments, the cell expresses the GCaMP7f and the red fluorescent protein. In some embodiments, the cell expresses the GCaMP7f and the fusion protein. In some embodiments, the cell is a target cell. In some embodiments, the cell is a mammalian cell. In some embodiments, the mammal is a human. In some embodiments, the cell is an in vivo cell. In some embodiments, the cell is an in vitro cell. In some embodiments, the cell is in an organism. In some embodiments, the organism is a mammal. In some embodiments, the cell is a neuron. In some embodiments, the cell is in a brain. In some embodiments, the neuron is a cortical neuron. In some embodiments, the neuron is a glutamatergic neuron. In some embodiments, the neuron is a cortical glutamatergic neuron.

By another aspect, there is provided a method of simultaneously fluorescently labeling a target cell and measuring calcium in the target cell, the method comprising expressing a nucleic acid molecule of the invention or an expression vector of the invention in the target cell, thereby simultaneously fluorescently labeling and measuring calcium in a target cell.

By another aspect, there is provided a method of analyzing a target cell, the method comprising:
a. receiving cells expressing a nucleic acid molecule of the invention or an expression vector of the invention;
b. isolating a cell comprising red fluorescence; and
c. performing an analysis on the isolated cell;
thereby analyzing a target cell.

In some embodiments, the method is an in vivo method. In some embodiments, the method is a method of in vivo phototagging. In some embodiments, the method is a method of in vivo phototagging and labeling. In some embodiments, the method is an ex vivo method. In some embodiments, the method is an in vitro method. In some embodiments, the target cell is a mammalian cell. In some embodiments, the mammal is a human. In some embodiments, the target cell is in an organ. In some embodiments, the target cell is in an organism. In some embodiments, the organism is a mammal. In some embodiments, the target cell is a neuron. In some embodiments, the target cell is in a brain. In some embodiments, the neuron is a cortical neuron. In some embodiments, the neuron is a glutamatergic neuron. In some embodiments, the neuron is a cortical glutamatergic neuron.

In some embodiments, the expression vector is formulated for expression in the target cell. In some embodiments, the at least one transcription regulatory element is active in the target cell. In some embodiments, active is active to induce transcription. In some embodiments, the at least one transcription regulatory element is specifically active in the target cell. In some embodiments, the promoter is a target cell specific promoter.

In some embodiments, the method further comprises shining an excitation light on the target cell. In some embodiments, the excitation light is a laser. In some embodiments, the excitation light photoconverts the photoactivatable red fluorescent protein. In some embodiments, the excitation light comprises wavelength of 810-850 nanometers (nm). In some embodiments, the excitation light comprises wavelength of 810-840 nm. In some embodiments, the excitation light comprises wavelength of 810 nm. In some embodiments, the excitation light is an equivalent light to one that comprises a wavelength of 810 nm and photoconverts the photoactivatable red fluorescent protein. In some embodiments, the excitation light excites GCaMP7f. In some embodiments, excites is induces fluorescence. In some embodiments, the excitation light comprises wavelength of 940 nm. In some embodiments, the excitation light comprises wavelength of 920-960 nm. In some embodiments, the excitation light is an equivalent light to one that comprises a wavelength of 940 nm and excites the GCaMP7f. In some embodiments, the excitation light photoactivates the photoactivatable red fluorescent protein. In some embodiments, the excitation light comprises wavelength of 1040 nm. In some embodiments, the excitation light is an equivalent light to one that comprises a wavelength of 1040 nm and photoactivates the photoactivatable red fluorescent protein. In some embodiments, a light of 810-840 nm is shined on the cell, a light of 940 nm is shined on the cell and a light of 1040 nm is shinned on the cell. In some embodiments, a light of 810-850 nm is shined on the cell, a light of 940 nm is shined on the cell and a light of 1040 nm is shinned on the cell. In some embodiments, a light of 810-840 nm is shined on the cell, a light of 920-960 nm is shined on the cell and a light of 1040 nm is shinned on the cell. In some embodiments, a light of 810-850 nm is shined on the cell, a light of 920-960 nm is shined on the cell and a light of 1040 nm is shinned on the cell. In some embodiments, all three lights are shined on the cell to excite the GCaMP7f, photoconvert the photoactivatable red fluorescent protein and photoactivate the photoactivatable red fluorescent protein.

In some embodiments, the light comprises a power of 37-42 milliwatts (mW). In some embodiments, the 810 nm light comprises a power of 37-42 mW. In some embodiments, the 810-840 nm light comprises a power of 37-42 mW. In some embodiments, the light comprises a power of at least 36 mW. In some embodiments, the light comprises a power of at least 37 mW. In some embodiments, the light is an 810 nm light. In some embodiments, the light is an 810-840 nm light. In some embodiments, the light is shone for a time sufficient for inducing excitation. In some embodiments, the time is sufficient to induce detectably fluorescence. In some embodiments, detectable fluorescence is at a resolution of at least 0.1 μm/pixel. In some embodiments, the time is at least 0.5 milliseconds per pixel (ms/pixel). In some embodiments, the time is at least 0.7 ms/pixel. In some embodiments, the time is between 0.7-1.3 ms/pixel. In some embodiments, the time is about 1.3 ms/pixel. In some embodiments, the time is at most 1.3 ms/pixel.

In some embodiments, the target cell is a functionally active cell. In some embodiments, the target cell is a functionally active neuron. In some embodiments, the target cell is Ca2+ positive. In some embodiments, the receiving is receiving a mixture of cells. In some embodiments, the mixture is a mixture of activated and non-activated cells. In some embodiments, the receiving is receiving tissue. In some embodiments, the tissue comprises activated cells. In some embodiments, all cells of the mixture comprise the nucleic acid molecule of the invention. In some embodiments, activated and non-activated cells of the mixture comprise the nucleic acid molecule of the invention. In some embodiments, all cells of the tissue comprise the nucleic acid molecule of the invention. In some embodiments, activated and non-activated cells of the tissue comprise the nucleic acid molecule of the invention.

In some embodiments, activated cells of the mixture or tissue comprise green fluorescence. In some embodiments, green fluorescence is when the cells are excited by a light with a wavelength of 446-494 nm. In some embodiments, green fluorescence is when the cells are excited by a light with a wavelength of 470 nm. In some embodiments, green fluorescence is detected from 474-575 nm. In some embodiments, green fluorescence is detected from 475-575 nm. In some embodiments, green fluorescence is detected with a band pass filter at 525+50 nm. In some embodiments, the receiving is receiving a mixture or tissue confirmed to have green fluorescence. In some embodiments, the mixture or tissue is a primary sample from an organism. In some embodiments, the organism is a mammal. In some embodiments, the mammal is a rodent. In some embodiments, the mammal is a human. In some embodiments, the organism is a laboratory animal.

In some embodiments, cells comprising red fluorescence are isolated. In some embodiments, at least 10, 15, 20, 25, 30, 35, 40, 45, 50, 55, 60, 65, 70, 75, 80, 85, 90, 92, 95, 97, 99 or 100% of cells with red fluorescence are isolated. Each possibility represents a separate embodiment of the invention. In some embodiments, red fluorescence is when the cells are excited by a light with a wavelength of 539-581 nm. In some embodiments, red fluorescence is when the cells are excited by a light with a wavelength of 561 nm. In some embodiments, red fluorescence is detected from 590-630 nm. In some embodiments, red fluorescence is detected from 600-630 nm. In some embodiments, red fluorescence is detected with a band pass filter at 610+20 nm and a 600 nm low pass filter.

In some embodiments, less than 10000, 5000, 4000, 3000, 2000, 1000, 900, 800, 750, 700, 600, 500, 400, 300, 250, 200, 100 or 50 cells are isolated. Each possibility represents a separate embodiment of the invention. In some embodiments, at most 1000 cells are isolated. In some embodiments, at least 1, 5, 10, 15, 20, 25, 30, 35, 40, 45, 50, 55, 60, 65, 70, 75, 80, 85, 90, 95 or 100 cells are isolated. Each possibility represents a separate embodiment of the invention. In some embodiments, at least 20 cells are isolated. In some embodiments, at least 50 cells are isolated. In some embodiments, at least 100 cells are isolated.

In some embodiments, isolated is sorted. In some embodiments, isolated is my FACS. In some embodiments, FACS is FACS sorting. Methods of sorting/isolating cells by fluorescence are well known in the art and can be performed to isolate the cells expressing red fluorescence.

In some embodiments, performing an analysis is performing a molecular analysis. In some embodiments, performing an analysis is performing a transcriptomic analysis. In some embodiments, performing an analysis is performing a proteomic analysis. In some embodiments, performing an analysis is performing a genomic or epigenomic analysis. In some embodiments, performing an analysis is performing sequencing. In some embodiments, sequencing is RNA-sequencing (RNA-seq). In some embodiments, the analysis is performed on RNA from the isolated cells. In some embodiments, the RNA is isolated from the cells. In some embodiments, the cells are lysed and the lysate is used for RNA-seq without RNA isolation. In some embodiments, the method is devoid of an RNA isolation step. In some embodiments, the analysis is performed on protein from the isolated cells. In some embodiments, a small number of cells are isolated and the RNA-seq is mesoscale RNA sequencing (Meso-seq). Standard RNA-seq is generally performed with thousands of cells, indeed usually with 100,000-1,000,000 cells. Meso-seq, in contrast, can be performed with as little as 20 cells/nuclei. In some embodiments, a small number is less than 1000 cells. In some embodiments, a small number is less than 800 cells. In some embodiments, a small number is less than 200 cells. In some embodiments, a small number is less than 100 cells. In some embodiments, a small number is 20-100 cells. In some embodiments, a small number is 20-200 cells. In some embodiments, a small number is 20-800 cells. In some embodiments, a small number is 20-1000 cells. In some embodiments, a small number is 50-100 cells. In some embodiments, a small number is 50-200 cells. In some embodiments, a small number is 50-800 cells. In some embodiments, a small number is 50-1000 cells. Methods of RNA-seq and Meso-seq are described herein and are known in the art. Further, the method of Meso-seq can be found in Apelblat et al., 2022, “Meso-seq for in-depth transcriptomics in ultra-low amounts of FACS-purified neuronal nuclei”, Cell Rep Methods, 2022 Jul. 25;2 (8): 100259, the contents of which are hereby incorporated by reference in their entirety). Any such method may be used for analysis of the isolated cells.

In some embodiments, the method further comprises lysing the isolated cells. In some embodiments, the method further comprises extracting RNA from the isolated cells. In some embodiments, the method further comprises extracting protein from the isolated cells. In some embodiments, the method further comprises extracting DNA from the isolated cells. In some embodiments, the extracting is isolating. In some embodiments, the extracting is purifying.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Wherever embodiments are described with the language “comprising,” otherwise analogous embodiments described in terms of “consisting of” and/or “consisting essentially of′ are included.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### Examples

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization-A Laboratory Course Manual” CSHL Press (1996); all of which are incorporated by reference. Other general references are provided throughout this document.

### Materials and Methods

Animals: All animal care and experiment procedures were in accordance with the guidelines of the National Institute of Health. Animal protocols were approved by the Columbia University Institutional Animal Care and Use Committee and the Weizmann Institute of Science Institutional Animal Care and Use Committee. Ad libitum water was provided until the beginning of training for the spatial navigation task.

Plasmids and Viral Constructs: pAAV-CW3SL-GCaMP7f-P2A-4Ala-H2B-PAmCherry was generated by standard cloning techniques. GCaMP7f (SEQ ID NO: 1) was PCR-amplified from Addgene plasmid #104492 with a 3′ Primer that contained sequences encoding the P2A sequence (SEQ ID NO: 7) and four Alanine residues (both in frame with the coding sequence of GCaMP7f). H2B-PAmCherry (SEQ ID NO: 24) was amplified from Addgene plasmid #133419. The PCR products were then subcloned by Gibson-assembly into Addgene plasmid #61463 after removing EGFP from this plasmid by restriction with Clal and EcoRI. The sequence of the cloned plasmid was validated by Sanger sequencing and GCaMP7f, P2A, 4xAla, and H2B-PAmCherry were all found to be in frame. The plasmid was packaged into AAVDJ at a viral titer of 7.26E+15.

Surgery: All procedures were performed with mice under anesthesia using isoflurane (4% induction, 1.5% maintenance in 95% oxygen). Mice's body temperature was maintained using a heating pad both during and after the procedure. Surgeries were performed on a stereotaxic instrument (Kopf Instruments). Before incision, mice were given subcutaneous meloxicam, as well as bupivacaine at the incision site. Doses were calculated based on the animal's weight. An incision above the skull was made to expose bregma and lambda for vertical alignment. Skull surfaces were cleaned and scored to improve dental cement adhesion. For viral injection, a glass capillary loaded with rAAV was attached to a Nanoject device (Drummond Scientific).

For all experimental mice, viruses were injected unilaterally in the left dorsal CA1 at 4 depths using the coordinates:−2.2 AP, −1.75 ML, and −1.2,−1.1,−1.0,−0.9 DV (relative to Bregma). At each depth, 75nl of AAVDJ-CW3SL-GCaMP7f-P2A-4Ala-H2B-PAmCherry was injected. After injection, surgical sites were closed with sutures. Three days after injection, the skull was exposed and a 3 mm craniotomy was made centered at the same coordinate of the injection site. Dura was removed, and the cortex was slowly aspirated with continuous irrigation of cold 1X PBS until the fiber tract above the hippocampus was visible. A 3-mm imaging cannula fitted with a 3 mm glass coverslip was implanted over the craniotomy site. Cannulas were secured by Vetbond. A custom titanium headpost for head-fixation was secured first with C&B Metabond (Parkell) and then dental acrylic. At the end of each procedure, the mice received a 1.0 ml saline injection subcutaneously and recovered in their home cage with heating applied. Mice were monitored for 3 days after the procedure.

Behavior paradigm: Mice were first water-deprived and habituated to handling and head fixation at least 7 days after implant surgery. They were then exposed to a 4-m long linear virtual reality (VR) corridor that stayed consistent in the training and recording. At the end of the environment, an inter-trial interval of 2 seconds of blank screen was included before the start of the next lap. For the next 10−14 days, mice were trained to run through the virtual environment and lick for a 5% sucrose reward. The rewards were first randomly distributed across the environment, and the number of rewards was slowly reduced from 30 at the beginning of the training to 2 when the mouse was deemed ready for recording. The final reward location was fixed toward the end of the VR environment. Mice were trained to run at least 30−60 laps in the environment. During behavioral imaging, mice were imagined during a single VR session (range: 18−40 min, 26+3 min, n=9 mice).

In vivo two-photon imaging and data processing: 2P functional imaging was conducted using an 8-kHz resonant scanner (Bruker) and a 16x near-infrared (NIR) water immersion objective (Nikon, 0.8 NA, 3.0-mm working distance). For population imaging, a field of view of 700 μm×700 μm was acquired at 30 Hz, 512×512 pixels using a 940-nm laser (Chameleon Ultra II, Coherent, 45−91 mW after the objective). Red (PAmCherry) and green (GCaMP7f) channels were separated by an emission cube set (green, HQ525/70 m-2p; red, HQ607/45 m-2p; 575dcxr, Chroma Technology), and fluorescence signals were collected with GaAsP photomultiplier tube modules (7422P-40, Hamamatsu). Following the acquisition of two-photon imaging data, Ca2+imaging data was structured and aligned with behavior data using the SIMA analysis package. CA1 ROIs were detected using the Suite2p (v0.14.2) package 86. To allow detection of all potential ROIs regardless of their activities during the recording. Suite2p was run with Cellpose (′anatomical_only′) for the ROI detection step. The pre-trained cyto2 model included in the published Cellpose package was used for ROI detection. When capturing two-photon z-stack images of photoactivated PAmCherry nuclei, a fixed wavelength 1070-nm laser (Fidelity-2W, Coherent) was used for excitation.

In vivo two-photon phototagging: Photoactivation was conducted using a three-dimensional random-access acousto-optical (3D-AOD) microscope (3D Atlas, Femtonics). Mice were head-fixed and anesthetized with isoflurane to minimize motion and increase the spatial precision of phototagging. The same 16x NIR water immersion objective was used to find the same field of view as in the functional recordings. Photoactivation was performed at 810-nm (Chameleon Ultra II, Coherent). Two-photon images of the mCherry red fluorescence were taken before and after photoactivation using a 1040-nm excitation laser (Alcor 1040−5W, Spark Lasers). Red (mCherry) and green (GCaMP7f) channels were separated by an emission cube set (green, HQ520/60 m-2p; red, HQ650/160 m-2p; 565dcxr, Chroma Technology), and fluorescence signals were collected with GaAsP photomultiplier tube modules (7422P-40, Hamamatsu). Two-photon images of the mCherry red fluorescence were taken before and after photoactivation using a 1040-nm excitation laser (Alcor 1040−5W, Spark Lasers). For photoactivation, a 7×7 μm, 0.1 μm/pixel scanning pattern was placed on the cell to be photoactivated. Each pixel was activated for a total dwell time of 1.3 ms with a laser power of 40 mW. This gave the total scanning time of each cell at 6,370 ms. Following photoactivation, a z-stack was taken for each mouse to assess the photoactivation efficacy.

For phototagging of ‘place cells’ and ‘silent cells’, photoactivation experiments were conducted a day after functional recording sessions. The imaging field of view of 700 μm×700 μm was matched between the 3D-AOD microscope and the time-averaged GCaMP image from functional recording. After confirming the same field of view as functional imaging, the viewport was zoomed in to a dimension of 250×250 μm for more effective identification of targeted cells according to the generated spatial masks. Following photoactivation of all cells, a z-stack image was taken for each mouse to confirm the tagging accuracy. Mice were given ad libitum water after functional imaging for at least 12 hours before photoactivation. During the session, mice were monitored every 10 min for breathing rate and reflexes. Heating and eye ointment were applied. Following the photoactivation, mice were returned to the home cage to recover with a heating pad.

Tissue dissociation and preparation of nuclei for FACS: For all animals used herein, tissue collection was performed at the same time of the day (5 pm) and at least one hour after the photoactivation. Mice were euthanized using CO2. The headpost and metal cannulas were removed. Dorsal CA1 of the hippocampus was collected by first using a 3-mm biopsy punch to cut a circular section of tissue the same size as the craniotomy. A microspatula was used to remove the shallow section of tissue that contained dorsal CA1. The tissue was placed in a 1.5 ml RNase-free Eppendorf tube and snap-frozen in liquid nitrogen. Tissues were stored at −80° C. until the start of the nuclei isolation.

To isolate the nuclei, each collected tissue was transferred to a dounce tissue homogenizer (DWK Life Sciences) with 1 ml of homogenization buffer (10 mM Tris Buffer, 250 mM Sucrose, 25 mM KCl, 5 mM MgCl2, 0.1 mM DTT, 0.1% Triton X-100, 1X Protease Inhibitor Cocktail, and 40U/μl RNAsin Plus RNase Inhibitor in nuclease-free water). Loose and tight pestles were used to break apart the tissue 10 times each. Following the homogenization, 1 ml of homogenization buffer was added to each dounce, and the homogenate was pipetted up and down to further break apart tissue before being passed through a 30 μm cell strainer (Miltenyi Biotec) and collected in a 15 ml conical tube.

The homogenate was centrifuged at 4° C., 700g for 8 min. The supernatants were then removed from the visible cell pellet. The cell pellet was resuspended with 800 μl of blocking buffer (1X PBS, 1% BSA, and 40 U/μl RNasin Plus RNase Inhibitor in nuclease-free water). The resuspension was incubated on ice for 15 min and transferred to a 1.5 ml tube. 2 μl of CoraLite Plus 488-conjugated NeuN Monoclonal antibody (Proteintech) was added to the resuspension, and incubated on an orbital rotator at 4° C. for 30 min. After incubation, nuclei were centrifuged at 4° C., 700 g for 8 min. The supernatant was removed, and the cell pellet was resuspended with 1000 μl of blocking buffer. DAPI was added to the suspension at a final concentration of 0.001 mg/ml, and the samples were passed through a 40-μm Flowmi Cell Strainer (Bel-Art). All samples were kept on ice until the start of FACS.

Fluorescence-activated cell sorting (FACS): The sorting was performed at the Zuckerman Institute Flow Cytometry Core using a MoFlo Astrios Cell Sorter (Beckman Coulter). Event rates were kept between 5000−10000 events per second. The cell sorter uses a linear array of lasers ordered as 640 nm, 488 nm, 561 nm, 532 nm, 405 nm and 355 nm from top to bottom. For experiments described herein, 488 nm, 561 nm and 405 nm lasers were used to detect the fluorescence of NeuN, mCherry and DAPI respectively. A gating control sample was used to set the gates for DAPI, NeuN and mCherry. Dissociated nuclei were passed through the cell sorter to collect those with high mCherry signals. Bright and Dim mCherry gates were determined after the first 5000 events of DAPI-positive nuclei. Gating was set to collect only the sparse and bright population that showed a high mCherry signal, and these events were collected as the photoactivated nuclei (‘Bright’ mCherry). Nuclei were collected into SMART-Seq CDS sorting buffer that contains 1X lysis buffer, SMART-Seq Oligo-dT and RNase inhibitor. All sorted samples were kept on dry ice as recommended by the SMART-Seq protocol until the start of first-strand synthesis. To increase the likelihood of collecting nuclei in this ultra-sparse population, aborted events for ‘Bright’ mCherry and all events for other positive mCherry were collected into the blocking buffer (200 ml). This suspension was passed through the sorter again with the same fluorescence gating following the completion of the first sorting to capture the bright nuclei.

Meso-seq: The previously published Meso-seq protocol (Apelblat, 2022) was followed with minor modifications. Sorted nuclei were collected in a lysis buffer following the SMART-Seq mRNA LP (with UMIs) protocol. Reverse transcription for cDNA was followed by cDNA amplification using 17−18 PCR cycles. Purified cDNA was prepared for sequencing the library using the SMART-seq Library Preparation Kit. Libraries were amplified using 14 PCR cycles. The concentration of the final library was determined using a Qubit3.0 Fluorometer (Invitrogen), and the average DNA fragment size was determined using a Bioanalyzer (Agilent). Sequencing was performed on a NextSeq 2000 sequencer with P2−100 reagents (Illumina). Libraries were diluted and pooled according to the recommendation of the sequencing kit.

Tissue collection and processing for immunohistochemistry and in situ hybridization: Mice were anesthetized with isoflurane and transcardially perfused with 20 mL of ice-cold 0.01M phosphate base saline (PBS, Sigma) followed by 20 mL ice-cold 4% paraformaldehyde (PFA, Electron Microscopy Sciences) in PBS. Brains were post-fixed in 4% PFA for 24 hours and then saturated with a 10%, 20%, and 30% sucrose solution sequentially over 48 hours until they sunk to the bottom of each successive solution. 30% Sucrose-saturated brains were then embedded in OCT (Optimal Cutting Temperature Compound, Sakura, cat #4583), frozen, stored overnight at −80° C., and sliced transversely at 20 μm thickness with a cryostat. Sections were stored at −80° C. on slides and used for either immunohistochemical staining or RNAScope in situ hybridization.

Immunohistochemistry: Sections (20 μm) were washed with PBS for 3×5 min. They were then permeabilized with a PBS and 0.3% Triton X-100 (Sigma-Aldrich) solution (PBST) for 2×20 min and blocked with a 10% NDS (Normal Donkey Serum, Jackson ImmunoResearch Labs) in PBST solution for 45 min. The sections were then incubated with a primary antibody for Calbindin (rabbit anti-Calbindin primary, 1:300, cat #: CB38a, Swant) diluted in PBST for 24 hours at 4° C. The sections were then brought to room temperature (RT) and washed with PBS for 3×5 min. Secondary antibodies (Donkey anti-rabbit Alexa-647, 1:300, Jackson Labs, cat #: 711−605−152) diluted in PBST with a 1:1000 dilution of DAPI (Sigma-Aldrich) to stain nuclei was then applied to the sections for 1 hour at RT. Sections were then washed with PBS for 3×5 min, and the sections were coverslipped and sealed with Fluoromount-GTM (Electron Microscopy Sciences, cat #: 17984−25). Sections were imaged in 3 μm z-steps using an inverted confocal microscope (A1 HD25, Nikon Instruments Inc; excitation filters: 405/488/568/647 nm).

RNAScope in situ hybridization: 20 μm fixed frozen sections of the frozen tissue block were taken and the RNAScope Multiplex Fluorescent Reagent Kit v2-User Manual was followed (cat #: 323100). The Calbindin targeting probe was designed and generated by Advanced Cell Diagnostics Inc (cat #: 428431, Entrez Gene ID: 12307, GenBank Accession #: NM_009788.4). Slides were sequentially dehydrated using ethanol solutions of increasing concentrations (50%, 70% and 100%) for 5 min at RT. 5−8 drops of H₂O₂ were added to each sample, followed by a 10 min incubation at RT. After rinsing with distilled water, antigen retrieval was performed for 5 min at 99° C. To digest sections, RNAscope Protease III was applied to the sections for 30 min at 40° C. The Calb1 probe was hybridized for 2 h at 40° C. and amplified with AMP1 (30 min), AMP2 (30 min), AMP3 (15 min); each was incubated at 40° C. The probe was fluorescently tagged with 1:2000 TSA Vivid Fluorophore 650 (PN 323273). Slides were counterstained with DAPI for a nuclear stain to identify viable Calbindin (−) cells and mounted in ProLong Gold Antifade Mountant. 20 μm sections were imaged in 3 μm z-steps using an inverted confocal microscope with 20x oil objective (A1 HD25, Nikon Instruments Inc.).

Data analysis

Quantification and Statistical Analysis: All statistical details for comparisons are described in the text. No statistical methods were used to determine sample sizes. Boxplots show the 25th, 50th (median), and 75th quartile ranges with the whiskers extending to 1.5 interquartile ranges below or above the 25th or 75th quartiles, respectively. Outliers are defined as values extending beyond the whisker ranges. For comparisons between two populations with non-normal distributions, the Mann-Whitney U test was used. For comparisons between gene expression datasets, the Wald test followed by multiple corrections via the Benjamini and Hochberg method was used as described in PyDESeq2.

Event detection: Fluorescence GCaMP traces were deconvolved using OASIS for fast nonnegative deconvolution89. As in ref. 90, these putative spike events were filtered at 3 median absolute deviations (MAD) above the raw trace, using a predetermined signal decay constant of 400 ms. The binarized signal was used to qualify whether a neuron was active at the respective frame. In the analysis, it is not claimed that true spiking events in these neurons are uncovered but rather the use deconvolution for denoising and diminishing Ca2+ autocorrelation.

Spatial tuning curves: The virtual environment was divided into 100 evenly spaced bins (4 cm), which were then utilized to bin a histogram of each cell's neuronal activity. Neuronal activity was filtered to include activity from when the animal was running above 3 cm/s and to exclude activity during the 2-see teleportation at the end of the 4-m track. The spatial tuning curves were normalized for the animal's occupancy and then smoothed with a Gaussian kernel (a=12 cm) to obtain a smoothed activity estimate.

Place/silent cell detection: Place fields were detected by identifying locations in the virtual environment where a neuron was more active than expected by chance. Each neuron's deconvolved spike trace was circularly shifted and the smoothed, trial-averaged spatial tuning curve of the shifted trace was recomputed to generate a shuffled null tuning curve per cell. This procedure was repeated 1000 times in order to calculate the 95th percentile of null tuning values at every spatial bin to generate a threshold for a p<0.05 significance curve. Spatial tuning curves that surpassed the null threshold were marked as candidate place fields, and the place field width was calculated as the total bins where the tuning curve exceeded the shuffled null tuning curve. To restrict the analysis to neurons with specific firing fields, it was additionally required that place fields have a width greater than 8 cm and less than one-third of the virtual environment (1.3 m). To ensure that the place field activity was stable, we also required that all place cells had activity for at least 20 laps.

If the binarized signal trace for a cell did not have any detected events via OASIS deconvolution, the cell was classified as silent.

PAmCherry fluorescence quantification: Red PAmCherry fluorescence was calculated as the tagged nuclei fluorescence versus the background fluorescence of nearby untagged nuclei for the image taken post-tagging. This was intentional to control for periodic two-photon imaging at 940 nm that may cause an increase in fluorescence for every cell in the FOV.

Fluorescence intensity distribution analysis: Red PAmCherry tagged nuclei were segmented using Cellpose with manual curation performed within the Cellpose GUI for the max axial projections of the ex vivo z-stack (excitation: 568 nm nm) and in vivo z-stack (1070 nm) to generate masks. The masks were used to segment the 3-D volumes of each cell and the respective fluorescence profiles were normalized and aligned based on the peak value. The average and standard error were computed based on the aligned fluorescence profiles of the cells from the respective in vivo and ex vivo volumes.

Two-photon background excitation fluorescence quantification: Changes in PAmCherry red fluorescence due to two-photon fluorescence excitation at 940 nm from functional recordings were examined. By taking the average fluorescence of the pre-imaging red channel image and post-imaging red channel image detected at 1070 nm, 11F/F was computed using the change in fluorescence between the average post-imaging red image and pre-imaging red image divided by the pre-imaging red image average [(post-pre)/pre].

Tagged and Off-target Fluorescence Change Quantification: Tagged nuclei were segmented using Cellpose with manual curation performed within the Cellpose GUI for the max axial projection of the in vivo-stacks from each mouse to generate masks. These masks were used to segment a 3D volume for each nucleus that was targeted across all mice. The number of off-target nuclei was manually quantified laterally and axially per targeted nuclei.

Masks for off-target nuclei were hand-drawn on the max axial projection to exclude the targeted nuclei and were at most 2 nuclei bodies away. Experimental background fluorescence masks were drawn on the surrounding areas with successfully tagged cells and excluded all targeted and off-target nuclei.

The fluorescence values for all tagged and off-target nuclei were percentile-filtered to exclude the lower 10% of fluorescence values to account for vignetting effects and mask inhomogeneities over the max axial projection. The percentile filtered fluorescence values for tagged and off-target nuclei were averaged, and the 11F/F was computed by taking the difference between the average tagged or off-target fluorescence value and the average background fluorescence and then normalizing by the average background fluorescence [tagged: (tagged-background)/background; off-target: (off-target-background)/b background]. Note this background value is distinct from the one described in the two-photon background excitation fluorescence quantification.

GCaMP quantifications: Frequency: The total number of deconvolved events by OASIS was normalized by the total duration of the recording.

For each cell, the average transient was segmented within a 15-second time window and computed by averaging along aligned deconvolved spike times. If multiple detected events were within 50 frames, the events were treated as a single transient. A cell was only used if there were at least 3 detected transients within the total trace to exclude cells without obvious GCaMP-Ca2+ dynamics. For each average transient, we computed the median value based on 5 seconds pre peak transient and the range 5−10 seconds after the peak transient (given that the average transient took significantly less than 5 seconds to resolve) to act as the baseline.

Amplitude: The difference between the max value of the average transient and the baseline value was computed Half-Rise Time: The time between the half-max value prior to the max and the max value of each transient was computed. We excluded cells where the average half-max value was not observed prior to the transient and performed 99th percentile filtering to remove extreme outliers. Due to sampling rate limitations, the true half-rise time cannot be commented on, so these are approximations.

Half-Decay Time: The time between the max and the half-max value was calculated following the transient peak. To remove extreme outliers, 99th percentile filtering was performed.

Image denoising: To correct for vertical scanning artifacts, combined wavelet and Fourier filters were utilized [github: github.com/DHI-GRAS/rmstripes]. Symlet 20 wavelets were used with varying levels of decomposition (2−5) for discrete wavelet transform to perform vertical striping correction in static images.

In vivo and ex vivo image registration: The in vivo and ex vivo images were transformed into 3D volumetric images for registration. The in vivo sequential images were concatenated across the z direction, i.e., depth, stacking the 2D images into a volumetric representation using MATLAB. The ex vivo images composed of 2D slices in each section were concatenated into 3D volumetric data using a stitching algorithm developed as a precursor for automatic ex vivo and in vivo registration [github:
github.com/ShuonanChen/multimodal_image_registration]. The discontinuity between ex vivo sections results in an unknown spatial correlation between them, requiring registration between sections. Common cells between sections (i.e., sections one and two) were used as reference markers for registration. The common cells were manually selected using a Napari GUI in Python and were utilized as reference markers to inform the scaling and affine transformations to be applied. The scaling and affine transformations were run automatically, transforming the second section to align with the first section. In the scaling transformation, the relative distances between cells in a section were compared to the cells in the first section, inducing an enlargement or shrinkage of the second section to match the first. In the affine transformation, the second section was geometrically transformed to align correctly with the first section. The transformations were obtained and applied to each slice in each section, and then each slice was concatenated together across the z direction to form a volumetric image.

The in vivo and ex vivo 3D images were then adjusted to have a uniform pixel size in all dimensions (1 μm in x,y and z), ensuring matching FOVs, and equivalent resolution across both images. Time-averaged representations of in vivo and ex vivo volumetric stacks were attained by employing maximum intensity projection (MIP) representations in FIJI, compressing the stacks into 2D images. The in vivo and ex vivo registration was carried out using a non-rigid registration algorithm for the (i) 3D volumetric stacks and (ii) MIP (2D) images. All cells common to both in vivo and ex vivo images were manually selected as the centroid of each cell using a Napari GUI in Python. The common cells were utilized as features to inform the scaling, affine transformation, and deformation transformation, which were applied to the ex vivo image. The scaling and affine transformations were run automatically. In the scaling transformation, the distances between the cells in the ex vivo image and matching cells in in vivo induce enlargement or shrinking of the ex vivo image to match the in vivo image. In the affine transformation, the ex vivo image was geometrically transformed to align with the in vivo image using the matching cells. The images in the GUI are updated to reflect the changes induced by the scaling and affine transformation. The deformation transformation uses a vector field, smoothed with Gaussian filtering, to move cells and deform the image, ensuring features in the transformed ex vivo image align with the in vivo image. The deformation transformation was iteratively employed, beginning with a Gaussian kernel size of 100, reducing to a kernel size of less than 10, with the user inspecting the alignment and making manual adjustments to the cell centroid position in the GUI. The completed transformed ex vivo image was then overlaid with the in vivo image.

RNA-sequencing data analysis: Sequencing data from the Illumina Sequencer was first post-processed through the Illumina DRAGEN secondary analysis pipeline to de-multiplex based on a unique index for each sample. RNA-seq reads were aligned to the mouse genome (mm39) using STAR. Unique reads were counted using HTSeq. HTSeq generated reads were then analyzed for differential expression using PyDESeq2. Following HTSeq counts, any genes with expression in less than 3 samples were discarded. FDR adjusted p values were used to determine significantly different genes. Both ‘place’ and ‘silent’ cell samples were analyzed against randomly tagged, function-blind, and sex-matched samples generated from mouse CA1 tissue in the same way as described in this methods section above. The same comparisons were made to identify differentially expressed genes for ‘place’ versus ‘random’, and ‘silent’ versus ‘random’. Within this ‘random’ dataset, male-female samples were compared to identify differentially expressed genes influenced by sex. All DEGs from these comparisons were cross-referenced to find common hits.

Gene set enrichment analysis: Gene set enrichment analysis was performed on HTSeq-generated counts using the gseapy package in Python 3.11. Enrichment of the fifty hallmark pathways from the molecular signatures database for Mus musculus (version 2023.2) was compared in place and silent cells. Comparisons were done with t-test and 1000 permutations.

### Example 1: In vivo two-photon phototagging with 2P-NucTag

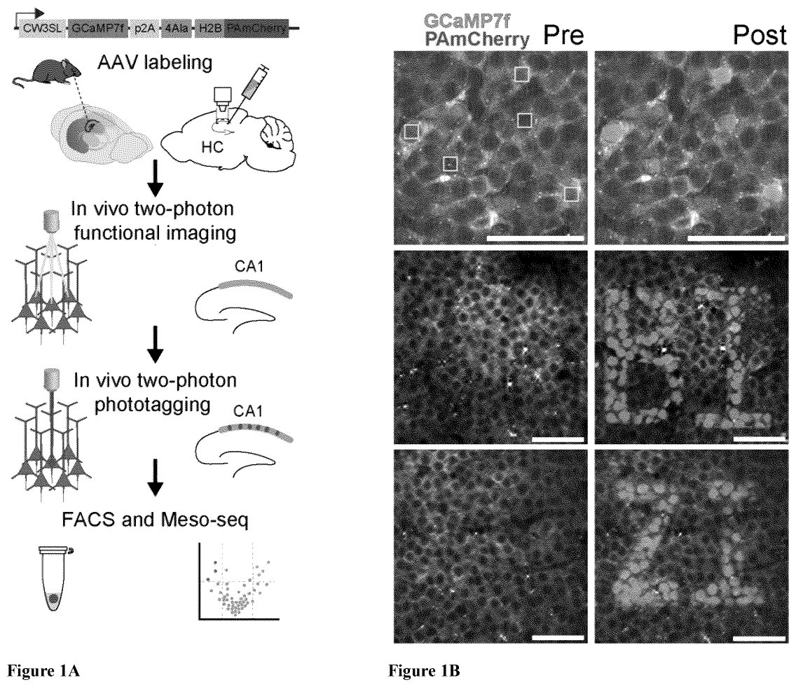

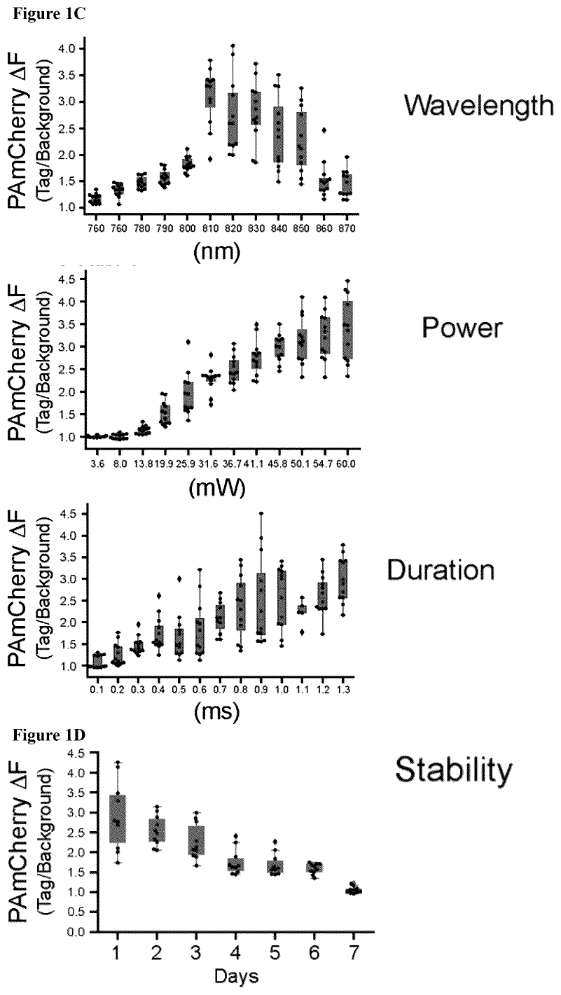

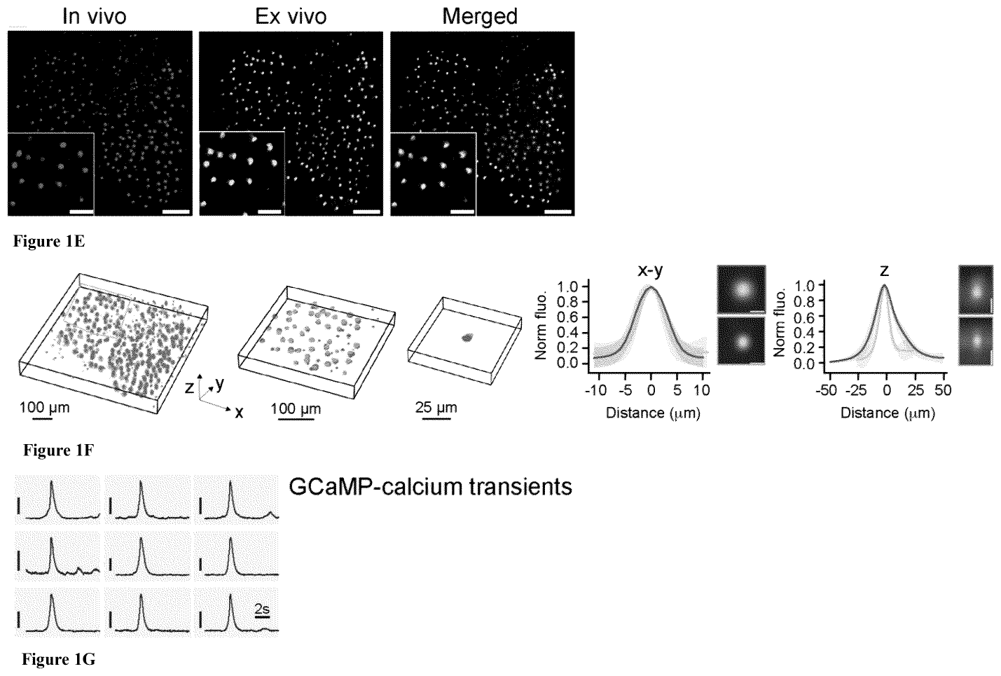

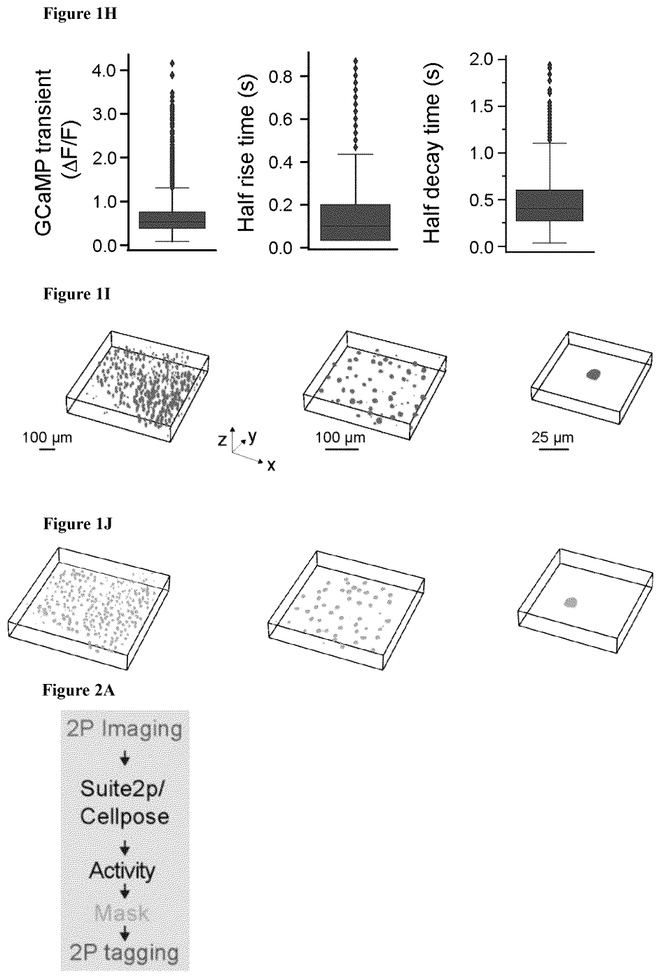

A major challenge in combining in vivo functional recording with stable tagging in the same neuron is the co-expression of an activity sensor and a photoactivatable tag with spectrally separable fluorescent imaging and photoactivation. This challenge was overcome by generating a bicistronic construct on a recombinant adeno-associated viral (rAAV) backbone that co-expresses cytosolic GCaMP7f for 2P Ca2+imaging and a nucleus-targeted photoactivatable red fluorescent protein (H2B-PAmCherry) for 2P phototagging under a promoter that is selective for cortical glutamatergic neurons (2P-NucTag, FIG. 1A). Upon injection of the 2P-NucTag rAAV into the CA1 region of the mouse dorsal hippocampus to label CA1 PNs, it was found that GCaMP7f is properly expressed in the perinuclear space of the infected PNs (FIG. 1B). Targeting the nuclei of GCaMP-expressing neurons, rapid nuclear PAmCherry photoconversion was achieved using 810-nm excitation light on a three-dimensional acousto-optical deflector microscope (3D-AOD) (FIG. 1B; see Methods) and orthogonal GCaMP-Ca2+ activity imaging with 940 nm excitation light (FIG. 1G-1H). Photoconverted PAmCherry red fluorescence was detected at >1000 nm excitation (1040 or 1070 nm, see Methods) and was localized to the targeted nuclei (FIG. 1B, top). 2P-NucTag enabled the imprinting of arbitrary tagging patterns into the CA1 pyramidal cell layer with 3D-AOD scanning, showing spatially precise photoconversion of H2B-PAmCherry with single-nuclear and even sub-nuclear resolution (FIG. 1B, middle and bottom). A detailed characterization of wavelength, laser power, and duration-dependence of PAmCherry photoactivation was carried out in vivo to identify the optimal parameters for spatially precise photoactivation (FIG. 1C). Based on the results, 810 nm excitation light with 37−42 mW laser power (measured after the objective), 1.3 ms/pixel dwell time over 70 pixel×70 pixel regions-of-interest (ROIs, with 0.1 μm/pixel resolution) was selected. The 810-nm wavelength is spectrally separated from the GCaMP-based Ca2+imaging wavelength at 940 nm, and these photoactivation wavelengths, laser power, and duration parameters yielded robust increases in PAmCherry fluorescence of targeted nuclei (192−379% increase in PAmCherry red fluorescence visualized using1040 nm excitation: n=36 cells, 295%=8% 11F/F, mean±s.e.m.) after single scans while minimizing total scan time and power. It was confirmed that phototagged nuclei remain detectable over multiple days after photolabeling (FIG. 1D). Next, a subset of CA1 PNs were photoactivated in a large (700×700 μm) field of view (FOV), similar to the FOV size used for in vivo 2P population imaging experiments in CA1 (FIG. 1E, left). Clear photoactivation of individual target nuclei were observed when visualized in vivo. It was then confirmed that in vivo photolabeling was preserved in post hoc histological slices (FIG. 1E, middle), and that phototagged cells can be reliably registered across in vivo z-stacks and post hoc confocal images (FIG. 1E, right, FIG. 1F, FIG. 1I-1J). In addition, the nuclei from both the in vivo and ex vivo z-stacks were segmented to observe the average axial and lateral fluorescence profiles of phototagged nuclei, demonstrating single-nucleus resolution (FIG. 1F, right). Thus, 2P-NucTag enables high-throughput, indelible phototagging of neuronal nuclei in vivo that can also be identified via a registration pipeline for post hoc analyses ex vivo.

### Example 2: Phototagging of functionally identified PNs in the hippocampus in vivo

To demonstrate the utility of 2P-NucTag for on-demand labeling of functionally defined PNs, 2P-NucTag was deployed to label hippocampal PNs located in the dorsal CA1 region which have well-established spatial coding heterogeneity. Only a subset of hippocampal PNs (‘place cells’) exhibit reliable spatial tuning for a location (‘place field’) during exploration. Place cell identity is thought to be randomly allocated within a seemingly homogenous population of PNs52, and is highly dynamic, with the majority of cells changing their spatial tuning properties over the timescale of days. The spatial intermingling of place cells with active-non-place and silent PNs53−56 without apparent topographical organization in the densely packed CA1 pyramidal layer allows for testing the utility of 2P-NucTag in selectively labeling PNs occupying these distinct functional states.

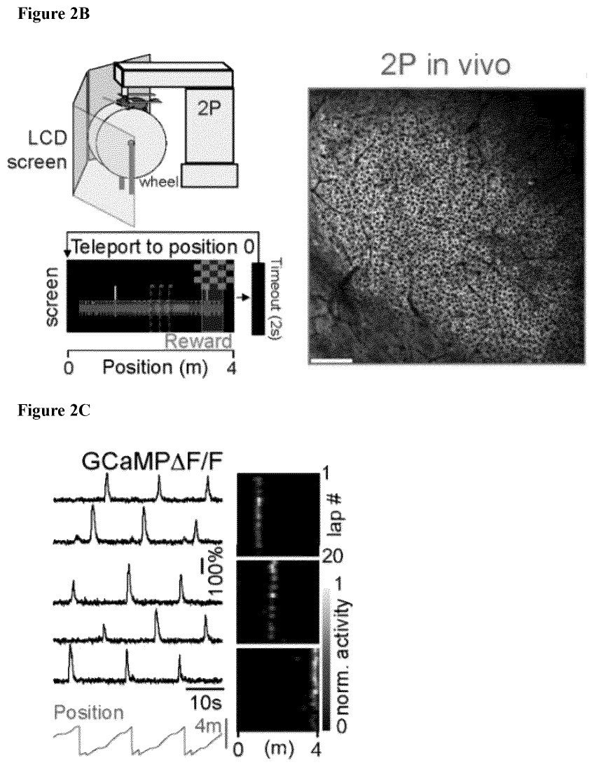

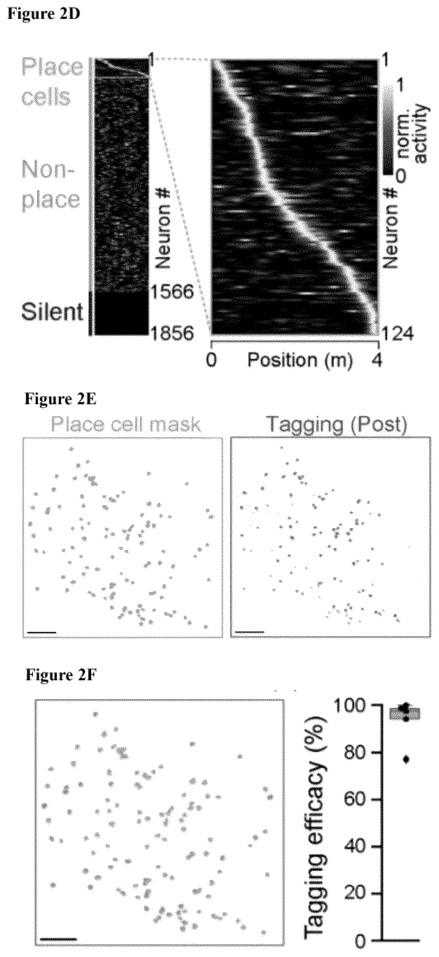

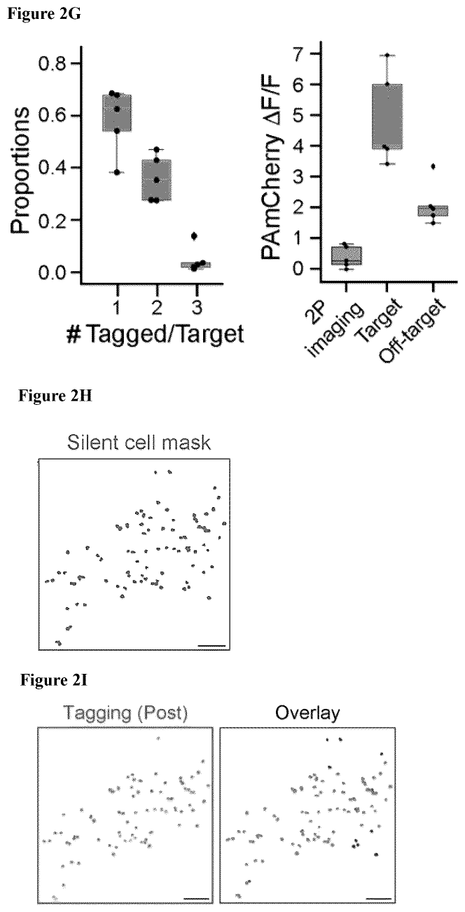

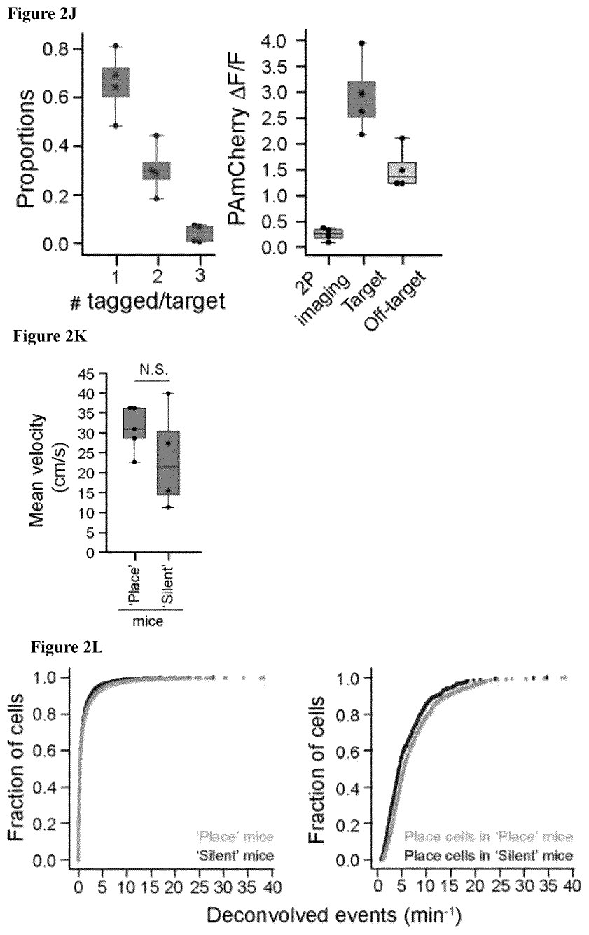

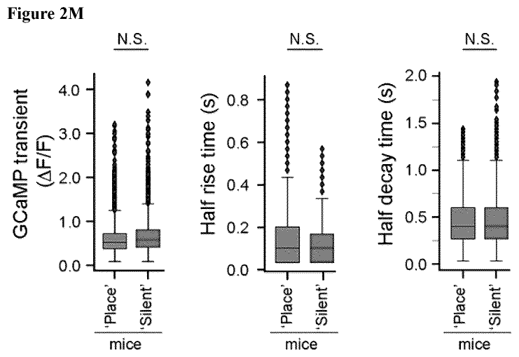

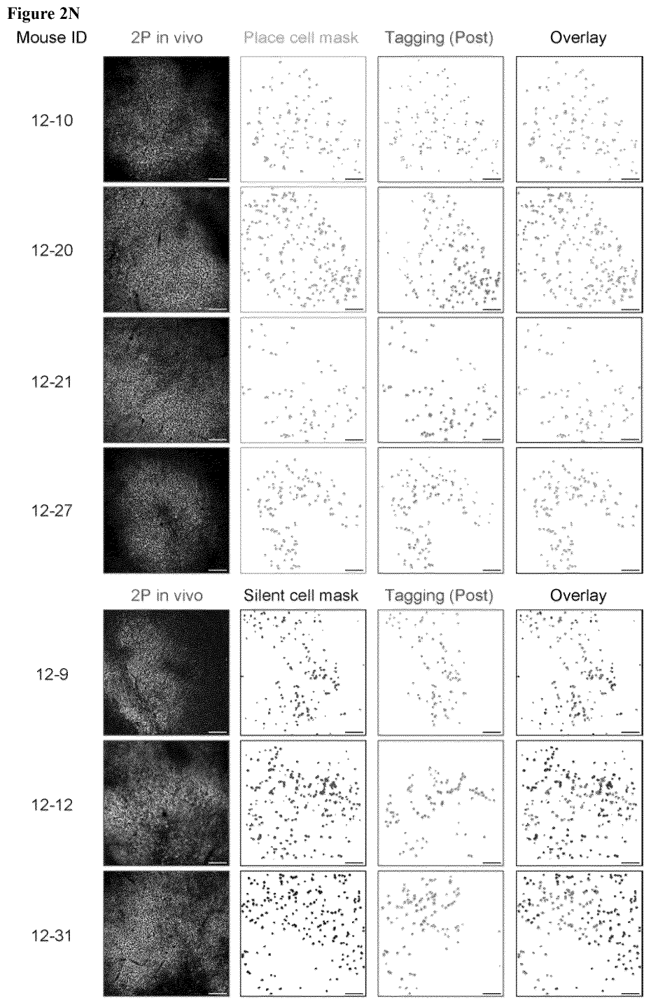

To selectively label CA1 PNs with distinct spatial coding properties, mice were trained in a spatial navigation task for water rewards in a linear virtual reality environment and in vivo 2P GCaMP-Ca2+imaging of PNs (FIG. 2A-2B) was performed. GCaMP-Ca2+ signals were reliably detected from individual PNs (FIG. 2C, 1I-1J). The basic characteristics of GCaMP7f in the bicistronic 2P-NucTag construct was analyzed and it was confirmed that the basic properties of the indicator are similar to those for a previously published single-construct version of GCaMP7f (Dana, et al., “High-performance calcium sensors for imaging activity in neuronal populations and microcompartments”, Nat Methods 16, 649−657 (2019), the contents of which are hereby incorporated by reference in its entirety) (FIG. 1I-J). All PNs in the imaging FOV were classified as place cells, active non-place cells, or silent cells (FIG. 2D, see Methods). Following the functional identification of PNs, spatial masks of place cell locations in the FOV were generated and these masks were used to guide 2P phototagging of all identified place cells in the imaging FOV with 3D-AOD (n=5 mice, FIG. 2E-F). In a separate set of mice (n=4 mice), a subset of CA1 PNs that exhibited no detectable activity during the imaging session were tagged (‘silent cells’ see Methods, FIG. 2H-M, Table 1). One could reliably register photoactivated nuclei to the spatial masks of functional profiles that were generated for cells of interest (FIG. 2F, 2H-I, 2N, see Methods). The number of place cell nuclei that we successfully photoactivated was quantified and compared to the number of spatial masks generated for each animal. Across all animals, 93.3%+4.2% (n=5 mice) of all targeted place cell masks were able to be phototagged (FIG. 2F). Aiming to tag approximately equal numbers of place and silent cells, an attempt was not made to tag all the silent cells, which are more abundant (Table 1).

TABLE 1

| | | | Place | Silent | Active | Mask | Tag |
|---|---|---|---|---|---|---|---|
| Mouse | Sex | Group | # | # | Non-Place # | # | # |
| 12-10 | Male | Place | 119 | 208 | 1754 | 119 | 112 |
| 12-20 | Female | Place | 250 | 470 | 1232 | 218 | 168 |
| 12-21 | Female | Place | 89 | 1040 | 701 | 74 | 73 |
| 12-27 | Female | Place | 132 | 547 | 548 | 119 | 119 |
| 12-37 | Female | Place | 126 | 290 | 1440 | 119 | 115 |
| 12-9 | Male | Silent | 9 | 152 | 655 | 152 | 87 |
| 12-12 | Male | Silent | 28 | 230 | 287 | 230 | 89 |
| 12-31 | Male | Silent | 129 | 305 | 1238 | 200 | 97 |
| 12-34 | Male | Silent | 187 | 303 | 571 | 113 | 79 |

To assess the accuracy of 2P photoactivation in the densely packed CA1 pyramidal layer, the number of tagged nuclei were quantified for each targeted cell, and it was found that photoactivation is well restricted to the target cells' nuclei with limited off-target labeled nuclei, which had a smaller increase in mCherry fluorescence (FIG. 2G, 2J). Furthermore, 2P GCaMP-Ca2+imaging at 940 nm over the course of the imaging session (18−40 min, see methods) resulted in a minimal increase of mCherry red fluorescence (37.4%=16.2% A1F/F, n=5 mice, FIG. 2G, 2J, 1070 nm excitation). Targeted place cells demonstrated higher mCherry fluorescence (487.7%+67% A1F/F, n=5 mice) compared to the background and off-target fluorescence (210.4%+32.1% A1F/F, n=5 mice). Mouse behavior and the quality of GCaMP recordings were consistent between the two groups of mice (silent and place, FIG. 2K-M). In sum, 2P-NucTag offers on-demand in vivo labeling of functionally defined neurons with high efficacy and accuracy.

### Example 3: Transcriptional profiling of functionally identified CA1 PNs

The ability to tag single cells in vivo enables a powerful new form of hypothesis generation and testing by which, for the first time, functionally defined cells with known behavioral relevance can be isolated and characterized. To demonstrate this, transcriptomic signatures of the functional ‘place’ and ‘silent’ cell states of CA1 PN were interrogated.

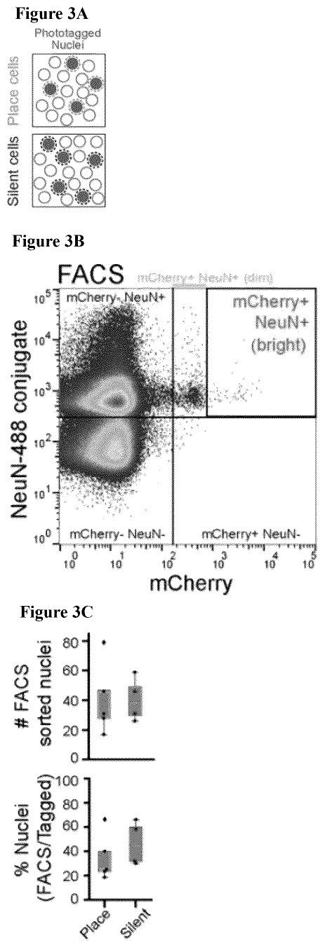

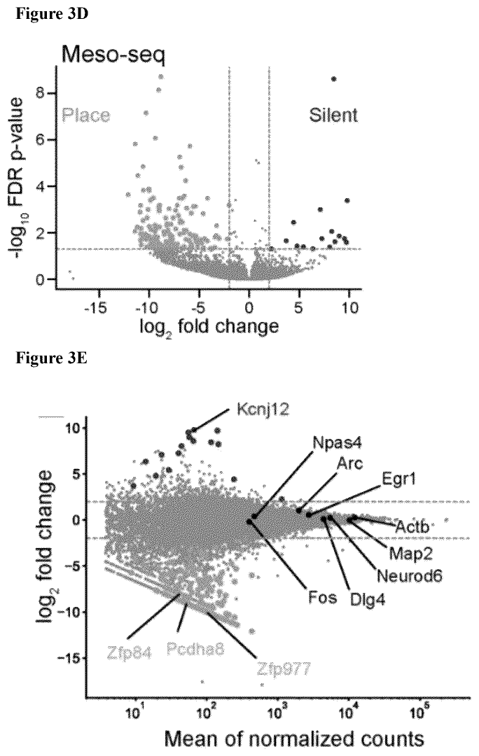

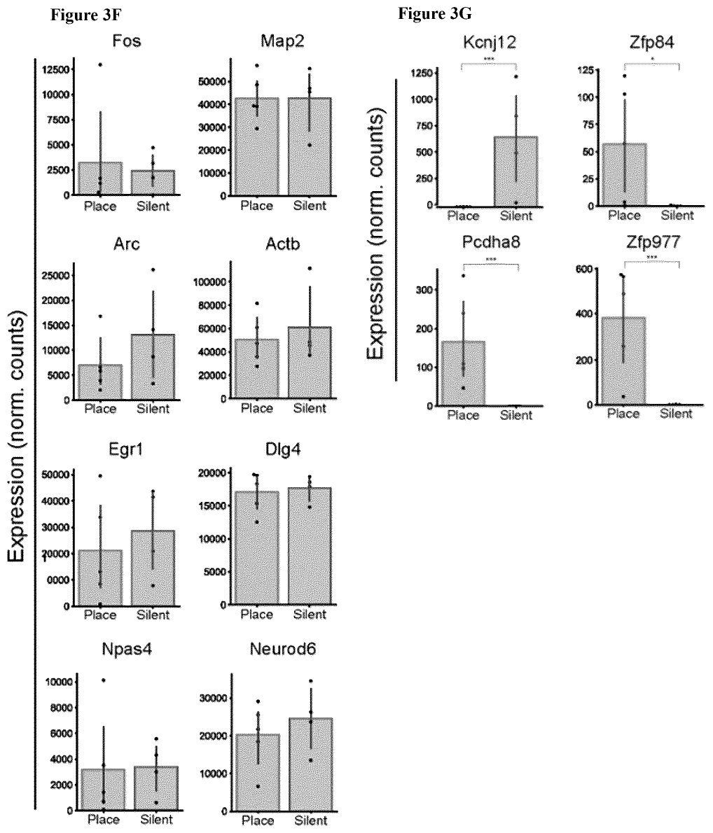

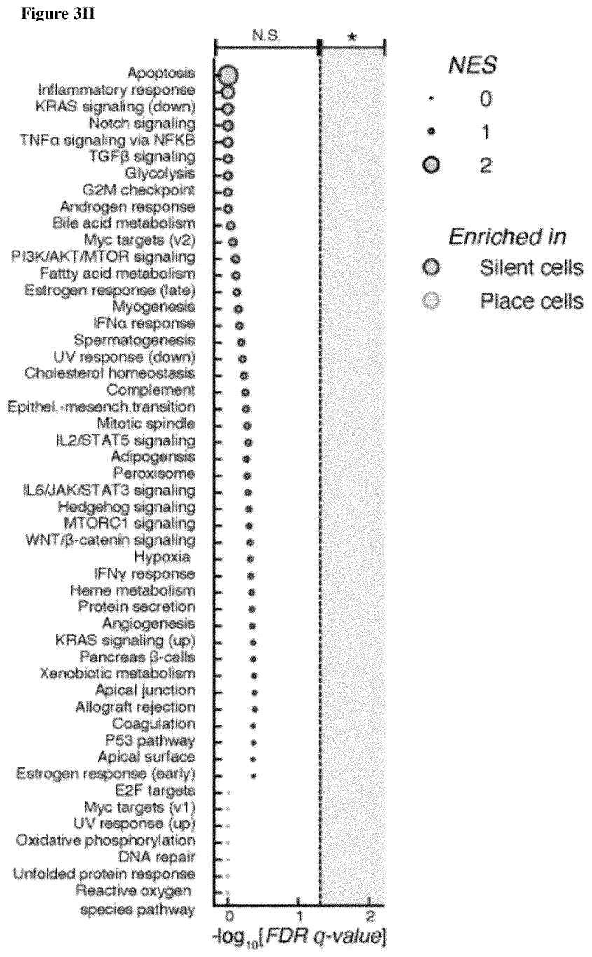

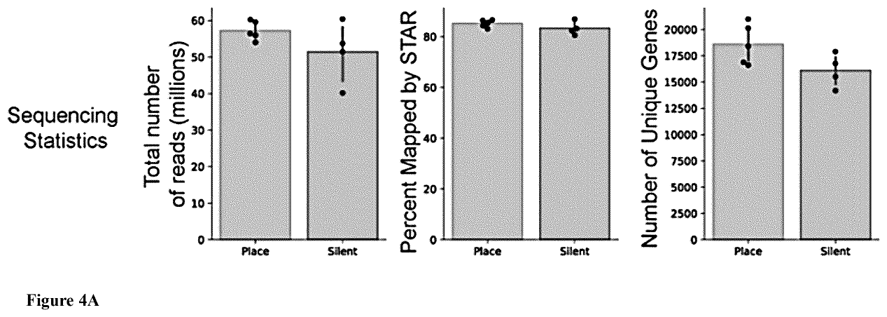

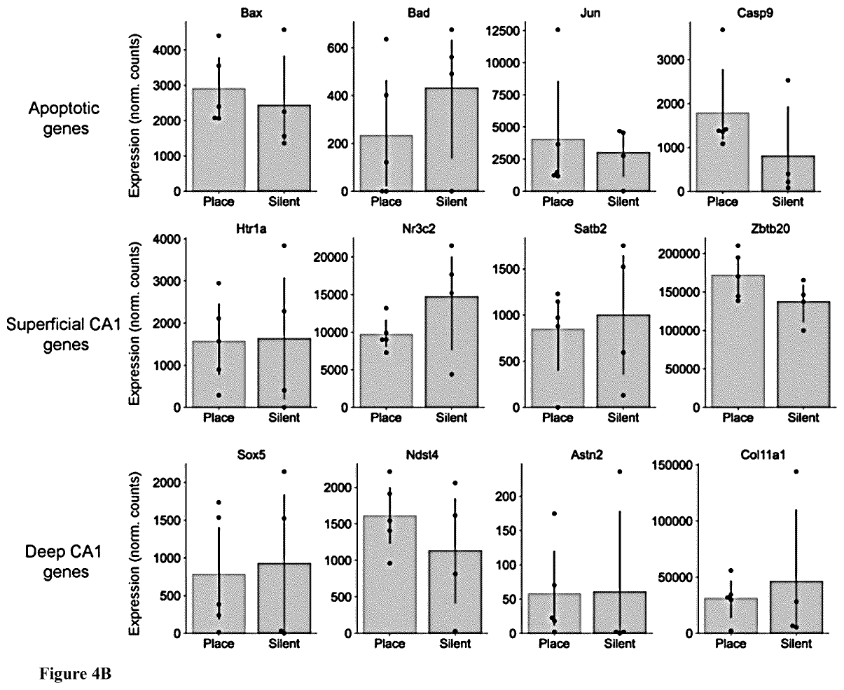

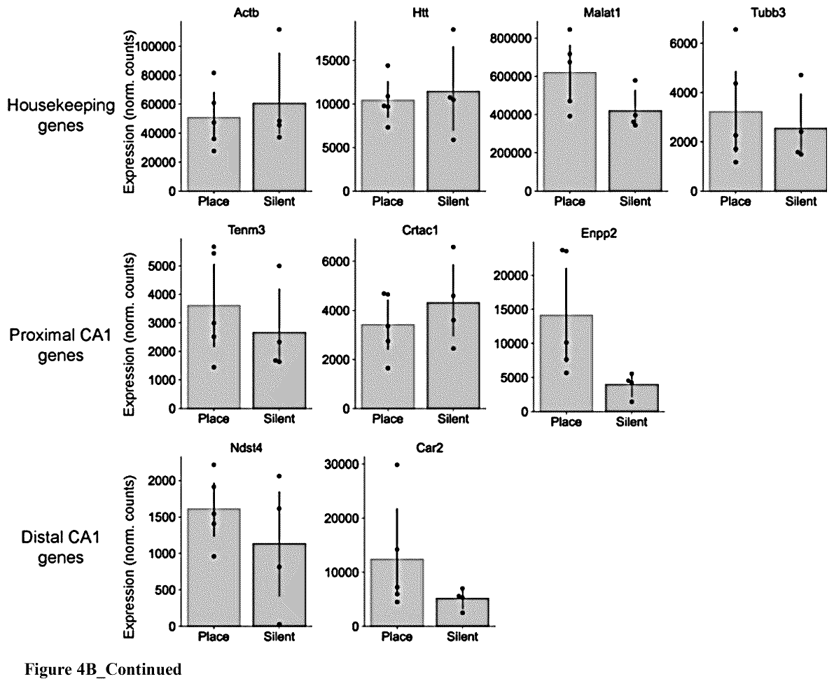

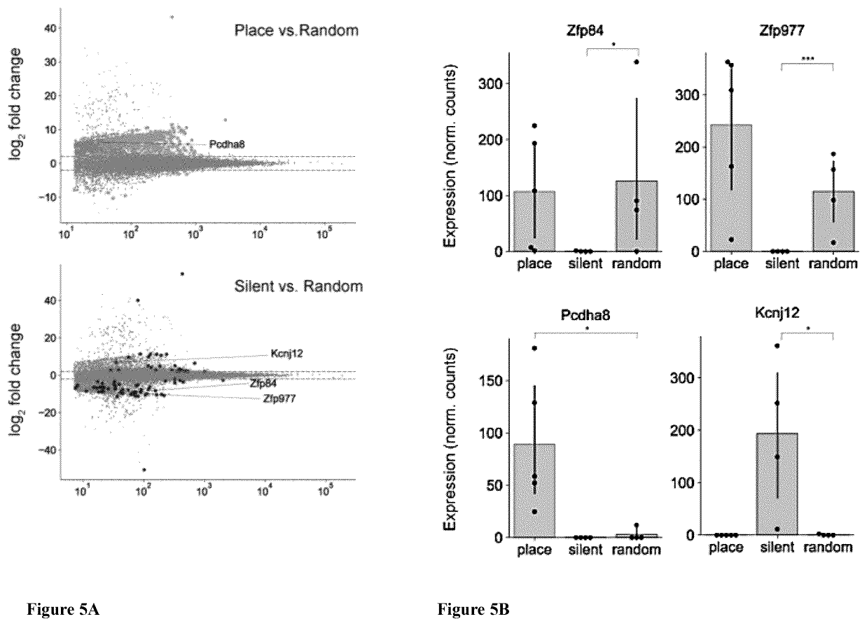

Brain tissue containing dorsal CA1 was collected, and nuclei were dissociated and stained with CoreLite 488-conjugated NeuN antibody and DAPI to identify neuronal nuclei (see methods). In vivo photoactivated nuclei were identified by bright mCherry fluorescence (FIG. 3A-3B) and were separated out from the non-photolabeled neuronal nuclei by fluorescent-activated cell sorting (FACS), enabling an estimated 18−67% recovery of all photoactivated nuclei identified during in vivo imaging (FIG. 3C). To determine whether place cells and silent cells differed in their gene expression programs, RNA-seq was performed on both populations of sorted nuclei by Meso-seq, an approach that enables reliable identification of differentially expressed genes in ultra-low amounts for FACS-isolated neuronal nuclei (i.e., tens of sorted nuclei, FIG. 3D). After FACS-isolating the photo-tagged nuclei, libraries were generated with the Meso-seq protocol and were sequenced at a depth of 40−60 million reads per library (FIG. 4A). Reads were aligned to the mm39 mouse genome assembly with STAR, counted with HTSeq, and gene expression patterns were compared between nuclei isolated from mice in which place cells were tagged (n=5) and mice in which silent cells were tagged (n=4) via PyDESeq2.

In both populations, canonical CA1 PN marker genes (Map2, Actb, Dlg4, Neurod6) were highly and non-differentially expressed, supporting the cellular precision of the tagging approach (FIG. 3E-3F). However, 219 genes were identified as differentially expressed in a significant manner between silent and place cells (FIG. 3E). In some cases, specific genes were reliably identified in one group and absent in the other. For example, no counts were measured for an inward rectifying potassium channel (Kcnj 12) in place cell samples, but the expression was present in all silent cell samples. Conversely, mRNA from two transcription factors of the zinc finger protein family (Zfp84, Zfp977) and a protocadherin gene (Pcdha8) were identified in every place cell sample and in none of the silent cell samples (FIG. 3G).

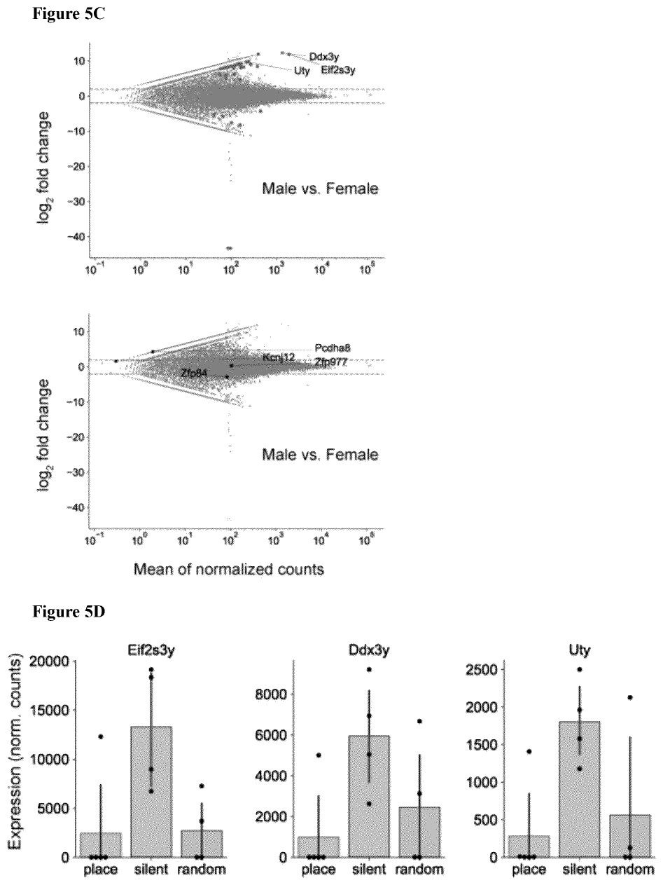

To ensure that the observed differences did not arise from other sources of variation between the place and silent cell samples, several additional analyses were performed, investigating potential contributions from anatomical positioning, transcriptional responses to neural activity, cellular health, and sex. Genes for which expression has been shown to vary along the dorsoventral and proximodistal axes of CA1 were not differentially expressed in the place and silent-cell nuclei (FIG. 4B), suggesting that any observed differences are unlikely to have originated from differences in anatomical positioning during cell tagging. Immediate early gene counts (Fos, Arc, Egr1, Npas4) did not differ (FIG. 3F), suggesting that the >25 hours between the last behavior session and tissue collection was sufficient time to eliminate the impact on the data of immediate transcriptional responses to neural activity. Apoptotic gene counts also did not differ between the two groups (FIG. 4A-4B), suggesting that there was no significant difference in cell health between the silent and place cells. Finally, to test for gross molecular differences between the cells in an unbiased fashion, gene set enrichment analysis was performed on fifty hallmark gene sets from the mouse molecular signatures database and no significant differences between silent and place cell samples was found (FIG. 3H). Since the sex of mice used for both groups was not well balanced in this study (Table 1), it is possible that gene expression differences originated from the mouse's sex rather than the functional identity of the tagged cells. To explore this possibility, gene expression in randomly tagged (function-blind) CA1 PN nuclei from both male (n=2) and female (n=2) mice were compared. Although it was found that known sex-specific genes were differentially expressed, none of the genes identified as enriched in either silent or place cells had significant sex associations (FIG. 5C-5D).

To assess if the differentially expressed genes-of-interest signified upregulation or downregulation in place cells from the mean or, conversely, downregulation or upregulation in silent cells, gene expression in place cells and silent cells was compared to randomly tagged cells separately. It was found that Kenj 12 expression was enriched in silent cells compared to random cells. Zfp84 and Zfp977 were significantly downregulated in silent cells compared to random cells, while Pcdha8 expression was significantly enriched in place cells compared to random cells (FIG. 5A-5B).

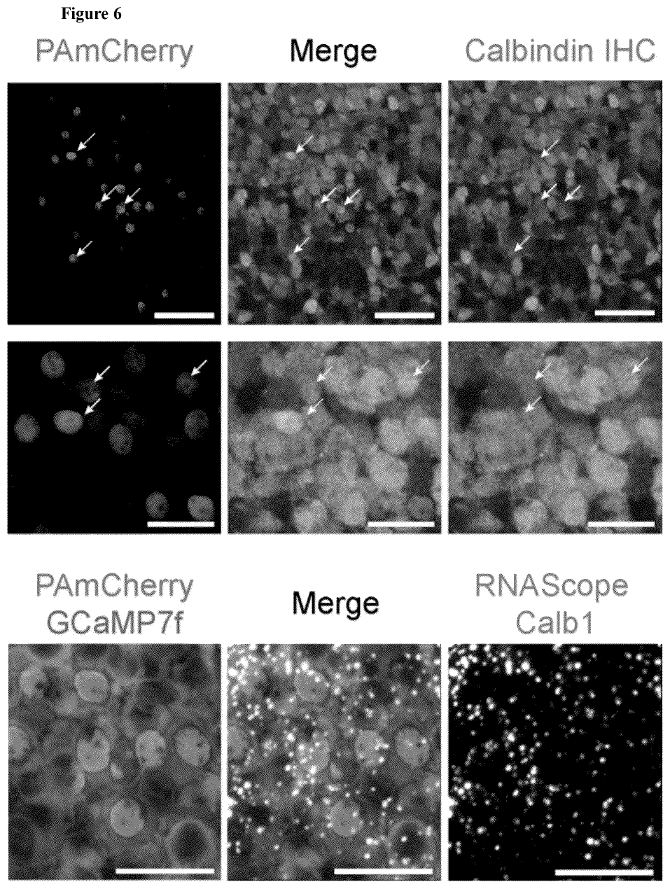

Finally, to confirm the compatibility of 2P-NucTag with other commonly used post hoc approaches for molecular analyses, immunohistochemistry and in situ hybridization were performed on dorsal CA1 tissues with randomly photoactivated nuclei (FIG. 6). Calbindin protein was immunolabeled and Calb1 transcripts were targeted using the RNAScope Multiplex Fluorescent assay, and it was found that 2P-NucTag signal is indeed stable and compatible with the PFA-fixed and fixed-frozen tissue processing commonly used for these imaging approaches.

In summary, the data demonstrate that, for the first time, researchers now have the ability to apply differential gene expression analysis on populations of cells from the same brain region that differ only by their functional identity. Moreover, this in-depth sequencing approach can indeed identify differences in gene expression between functionally defined cells tagged in vivo with 2P-NucTag.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A nucleic acid molecule comprising at least one transcription regulatory element operably linked to an open reading frame, wherein said open reading frame encodes a single RNA transcript encoding GCaMP7f, a ribosomal skipping peptide, and a fusion protein of a nuclear protein and photoactivatable red fluorescent protein.

2. The nucleic acid molecule of claim 1, wherein said GCaMP7f comprises the amino acid sequence
(SEQ ID NO: 2)
MGSHHHHHHGMASMTGGQQMGRDLYDDDDKDLATMVDSSRRKWNKTG
HAVRAIGRLSSLENVYIKADKQKNGIKANFKIRHNIEDGGVQLAYHYQQ
NTPIGDGPVLLPDNHYLSVQSKLSKDPNEKRDHMVLLEFVTAAGITLGM
DELYKGGTGGSMVSKGEELFTGVVPILVELDGDVNGHKFSVSGEGEGDA
TYGKLTLKFICTTGKLPVPWPTLVTTLTYGVQCFSRYPDHMKQHDFFKS
AMPEGYIQERTIFFKDDGNYKTRAEVKFEGDTLVNRIELKGIDFKEDGN
ILGHKLEYNLPDQLTEEQIAEFKELFSLFDKDGDGTITTKELGTVMRSL
GQNPTEAELQDMINEVDADGDGTIDFPEFLTMMARKMKYTDSEEEIREA
FRVFDKDGNGYISAAELRHVMTNLGEKLTDEEVDEMIREADIDGDGQVN
YEEFVQMMTAK.

3. The nucleic acid molecule of claim 1, wherein said ribosomal skipping peptide is selected from P2A, T2A, E2A and F2A, optionally wherein a sequence encoding GSG directly 5′ to a sequence encoding said ribosomal skipping peptide.

4. The nucleic acid molecule of claim 3, wherein said ribosomal skipping peptide is P2A comprising the amino acid sequence ATNFSLLKQAGDVEENPGP (SEQ ID NO: 8).

5. The nucleic acid molecule of claim 1, wherein said photoactivatable red fluorescent protein is a PamCherry protein.

6. The nucleic acid molecule of claim 5, wherein said PAmCherry protein is PamCherryl and comprises the amino acid sequence:
(SEQ ID NO: 4)
MVSKGEEDNMAIIKEFMRFKVHMEGSVNGHVFEIEGEGEGRPYEGTQTA
KLKVTKGGPLPFTWDILSPQFMYGSNAYVKHPADIPDYFKLSFPEGFKW
ERVMKFEDGGVVTVTQDSSLQDGEFIYKVKLRGTNFPSDGPVMQKKTMG
WEALSERMYPEDGALKGEVKPRVKLKDGGHYDAEVKTTYKAKKPVQLPG
AYNVNRKLDITSHNEDYTIVEQYERAEGRHSTGGMDELYK.

7. The nucleic acid molecule of claim 1, wherein said nuclear protein is a histone.

8. The nucleic acid molecule of claim 7, wherein said histone is Histone 2B (H2B), optionally wherein said H2B is H2B type 1-J and comprises the amino acid sequence
(SEQ ID NO: 16)
MPEPAKSAPAPKKGSKKAVTKAQKKGGKKRKRSRKESYSIYVYKVLKQV
HPDTGISSKAMGIMNSFVNDIFERIAGEASRLAHYNKRSTITSREIQTA
VRLLLPGELAKHAVSEGTKAITKYTSAK.

9. The nucleic acid molecule of claim 1, wherein
a. said fusion protein comprises a peptide linker between said histone and said photoactivatable red fluorescent protein, and wherein said peptide linker is between 1 and 10 amino acids is length;
b. said molecule further comprises a linker sequence encoding an amino acid linker between said ribosomal skipping peptide and said fusion protein, wherein said amino acid linker is 4−10 amino acids in length, optionally wherein said amino acid linker comprises four consecutive alanine residues; or
c. both.

10. The nucleic acid molecule of claim 1, wherein said single RNA transcript encodes SEQ ID NO: 22, wherein said open reading frame comprises SEQ ID NO: 21 or both.

11. The nucleic acid molecule of claim 1, wherein said transcription regulatory element is a promoter.

12. The nucleic acid molecule of claim 11, wherein said promoter is a CAMKII promoter or a fragment thereof that drives transcription in cortical glutamatergic neurons.

13. The nucleic acid molecule of claim 12, wherein said CAMKII promoter or fragment thereof comprises
(SEQ ID NO: 23)
cacttgtggactaagtttgttcgcatccccttctccaaccccctcagta
catcaccctgggggaacagggtccacttgctcctgggcccacacagtcc
tgcagtattgtgtatataaggccagggcaaagaggagcaggttttaaag
tgaaaggcaggcaggtgttggggaggcagttaccggggcaacgggaaca
gggcgtttcggaggtggttgccatggggacctggatgctgacgaaggct
cgcgaggctgtgagcagccacagtgccctgctcagaagccccaagctcg
tcagtcaagccggttctccgtttgcactcaggagcacgggcaggcgagt
ggcccctagttctgggggcagc.

14. An expression vector comprising the nucleic acid molecule of claim 1, optionally wherein said expression vector is an adeno-associated viral vector (AAV).

15. A cell comprising a nucleic acid molecule of claim 1.

16. A method of simultaneously fluorescently labeling and measuring calcium in a target cell, the method comprising expressing a nucleic acid molecule of claim 1 in said target cell, thereby simultaneously fluorescently labeling and measuring calcium in a target cell.

17. The method of claim 16, being an in vivo method.

18. The method of claim 16, further comprising shinning on said target cell an 810−840 nm excitation light or an equivalent light that photoconverts said photoactivatable red fluorescent protein, a 920−960 nm excitation light or an equivalent light that excites said GCaMP7f, and a 1040 nm excitation light or an equivalent light that excites the photoactivated photoactivatable red fluorescent protein.

19. A method of analyzing a target functional active cell, the method comprising,
a. receiving a mixture of activated cells and non-activated cells or a tissue comprising activated cells expressing a nucleic acid molecule of claim 1 and comprising green fluorescence detected at 474−575 nm;
b. isolating a cell comprising red fluorescence detected at between 600−630 nm; and
c. performing RNA-sequencing (RNA-seq) on RNA from said isolated cell;
thereby analyzing a target functionally active cell.

20. The method of claim 19, wherein a single cell is isolated and said RNA-seq is single cell RNA-seq or wherein 20−200 cells are isolated and said RNA-seq is mesoscale RNA sequencing (Meso-seq).
